(19) United States
(12) Patent Application Publication
(10) Pub. No.: US 2024/0368148 A1
(43) Pub. Date: Nov. 7, 2024

(54) DIFUNCTIONAL BIO-BENZOXAZINE COMPOUNDS, PREPARATION METHODS AND USES THEREOF

(71) Applicant: Paul JONES, Wrightington (GB)

(72) Inventor: Paul JONES, Wrightington (GB)

(21) Appl. No.: 18/687,675

(22) PCT Filed: May 5, 2022
(86) PCT No.: PCT/GB2022/051146

§ 371 Date: Feb. 28, 2024

(30) Foreign Application Priority Data
Sep. 1, 2021 (GB) 2112469.8

(51) Int. Cl. C07D 413/14; C08G 63/685; C08G 63/82; C08J 5/24

(52) U.S. Cl. CPC C07D 413/14; C08G 63/685; C08G 63/826; C08J 5/243; C08J 5/244; C08J 2367/00

(57) ABSTRACT

The invention relates to a class of difunctional benzoxazine compounds useful as curatives, as well as methods of preparation, and uses thereof. The difunctional benzoxazine compounds comprise two benzoxazine rings, each disubstituted with two furfuryl groups. The two benzoxazine rings may be attached via a hydrocarbyl linker or two benzoxazine moieties may share a common benzene group. The difunctional benzoxazine compounds are capable of self-polymerisation via a ring opening reaction which allows them to act as a curative. In one aspect, the present invention provides a compound of formula (I) below. (I)

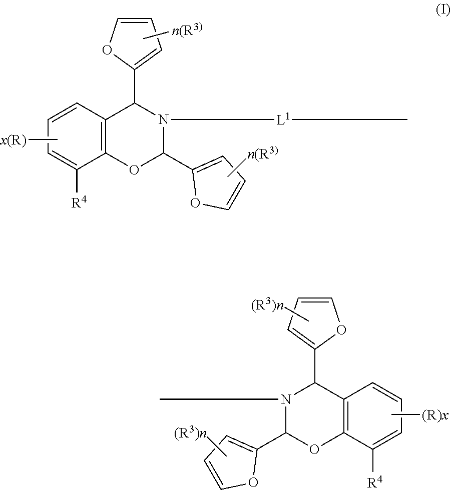

The invention relates to a class of benzoxazine compounds useful as a curative and having a high degree of biological based carbon content. In particular the invention is directed to a class of difunctional benzoxazine compounds comprising two benzoxazine rings, each di-substituted with furfuryl groups, a resin curative composition comprising the benzoxazine compound, processes for preparing the benzoxazines and uses thereof.

### BACKGROUND OF THE INVENTION

A benzoxazine is chemical compound comprising a bicyclic ring system containing an oxygen atom and a nitrogen atom within a heterocyclic oxazine ring that is directly fused to a benzene ring. There are several isomers of benzoxazine with the nitrogen and oxygen at differing positions that may be generically referred to as “benzoxazines”, in addition to compounds having differing levels of saturation in the oxazine ring that may also go by the name “benzoxazine”. In this application, the term “benzoxazine” refers to 3,4-dihydro-2H-1,3-benzoxazine, which is the benzoxazine monomer most useful in the production of benzoxazine based polymers, the structure of which is shown below.

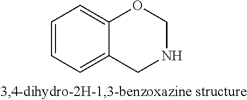

Benzoxazines are capable of undergoing ring opening on heating without the need of catalysts and without emitting volatiles, leading to self-polymerisation, which is useful for both providing thermosets and also in curing to reinforce other materials. The benzoxazine polymers formed therefrom are the products of interest for providing suitable molecular weight structures that confer desirable protective properties once fully cured.

The synthesis of benzoxazine dates back over 60 years although the low molecular weight species at that time could not offer the performance characteristics now attributed to, and associated with, the polymeric benzoxazines that form the basis of the coating and matrix systems employed today. Commercially available benzoxazines are generally provided by reaction of i) a phenolic compound, which may be phenol, bisphenol, or a substituted derivative thereof, ii) formaldehyde which comes in different concentrations as an aqueous solution of the gas or in polymerized form as paraformaldehyde, and iii) a primary amine which contains one or multiple primary amine groups, but may contain additional secondary or tertiary amine groups and/or other functional groups.

The process to synthesise the benzoxazine structure is a Mannich reaction between the primary amine and the aldehyde liberating water to form an iminium ion, followed by cyclisation with the phenolic compound and a second equivalent of aldehyde to form the oxazine ring. The Mannich reaction to form a benzoxazine ring, followed by self-polymerisation, is shown in Scheme 1 below.

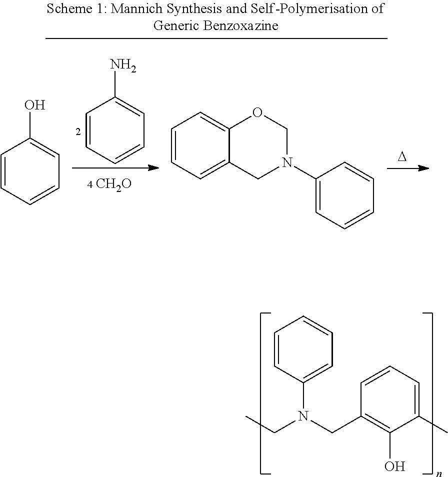

The most desirable benzoxazine polymers for coating and matrix applications are derived from difunctional benzoxazines. The resultant products have repeating units of a higher molecular weight and form polymers with a higher degree of cross-linkage. A difunctional benzoxazine may also comprise only one benzene ring that is fused to two separate oxazine rings, providing two benzoxazine moieties on a shared benzene ring.

For this reason, bisphenol A is a well-known choice of phenolic compound for this purpose as it comprises two phenol groups capable of transformation into benzoxazines. Cardarez R111 (RTM), depicted below, is a known bisphenol A based benzoxazine useful for providing polymers with a high glass transition temperature (Tg), low water absorption, excellent physical electrical performance, along with excellent fire resistance properties. Like other conventional benzoxazines, Cardarez R111 (RTM) forms a solid resin when prepared.

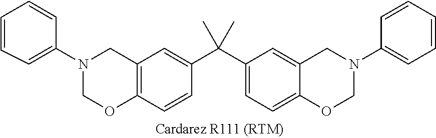 [SMILES: CC(C)(c1ccc2c(c1)CN(c1ccccc1)CO2)c1ccc2c(c1)CN(c1ccccc1)CO2]

However, a disadvantage of conventional benzoxazines, is that the formaldehyde from which they are prepared is a highly toxic and carcinogenic chemical that is synthesised by energy intensive industrial processes. Furthermore, as mentioned above, benzoxazines formed by these methods tend to be solid resins. The process temperature and the melting point of the product also results in increased viscosity and subsequent difficulty in management or the process. In particular, increased viscosity can also generate problems when processing as a solvent free matrix or coating system. Furthermore, conventional benzoxazine resins often suffer from slow gel times, reducing the efficiency of processes such as the formation of matrixes or coatings.

The present invention is based on the surprising discovery of a new class of difunctional benzoxazine compounds that are liquids under standard conditions (e.g. at 25° C. and 100 KPa), but provide thermosets with comparable properties, such as viscosity and Tg, whilst also providing greatly increased bio-carbon content and obviating the need for synthesis from formaldehyde. Resin compositions formed from the new class of difunctional benzoxazine compounds of the present invention also present superior gel times, converting to a solid material more rapidly than conventional benzoxazine resins.

The bio-carbon content of the benzoxazines of the present invention is greatly increased by replacing the formaldehyde precursor with furfuraldehyde, also known as furfural. Furfural is an inexpensive, renewable and naturally occurring feedstock derived from the dehydration of sugars, and occurs in a variety of agricultural by-products, including corncobs, oats, wheat bran, and sawdust.

Furfural has been used in the synthesis of aryloxazines in the pharmaceutical industry, in particular, napthoxazines have been investigated for potential medicinal properties. See M. S. Al Ajely and A M Noori, Synthesis of New Oxazin Compounds Derived from Furfural. Chalcons and Schiff base, LOJ pharmacol., 2019, 1(3), 66-71. and S. A. Ozturkcan and T Zuhal, Synthesis and Characterizations of New 1,3-Oxazine Derivatives, J. Chem. Soc. Pak., 2011, 33(6), 939-944.

D. R. de Oliveira, et al, Synthesis and Polymerization of Naphthoxazines Containing Furan Groups: An Approach to Novel Biobased and Flame-Resistant Thermosets, Int. J. Polym. Sci., 2018, 2018, 1-13, describe the use of solid furfuryl substituted napthoxazines, prepared from furfurylamine or furaldehyde precursors, as monomers for the production of polymeric materials. However, the polymer repeating unit of this disclosure is formed from a single napthoxazine ring.

The difunctional benzoxazine of the present invention is useful in forming a polymer comprising repeating units formed from a difunctional benzoxazine to provide a high degree of cross-linkage and a high bio-carbon based content. Surprisingly, the new class of furfural derived difunctional benzoxazines of the present invention have also been found to typically be in liquid form at standard conditions (e.g. 25° C. and 100 kPa), which provides numerous advantages in handling, application and use in resin compositions over conventional solid benzoxazine resins. For example, liquid benzoxazines may be combined with a curing catalyst at low temperature (below which reaction of the benzoxazine can be expected) without the risk of unwanted or premature self-polymerisation. In contrast, a solid benzoxazine compound such as Cardarez R111 (RTM) must be melted before mixing with a catalyst is possible, thus risking undesired self-polymerisation due to the high temperature required for melting.

### SUMMARY OF THE INVENTION

In one aspect, the present invention provides a compound of formula (I):

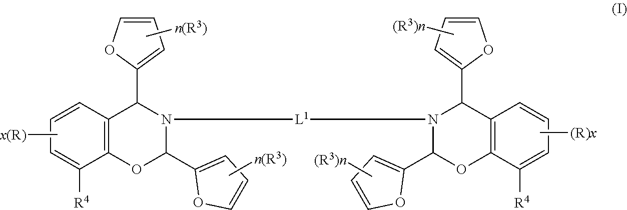

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein L¹ is a divalent hydrocarbyl linker comprising 1 to 50 carbon atoms;
wherein x is independently 0, 1, 2 or 3; and
wherein n is independently 0, 1, 2 or 3.

In another aspect, the present invention provides a compound of formula (II):

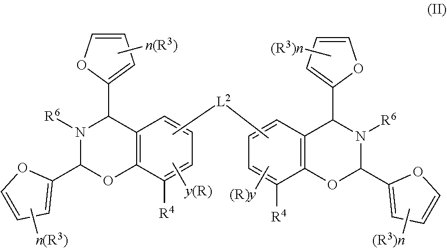

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein L² is a direct bond, a divalent group selected from: C(O), O, S, S(O), S(O)₂, or a hydrocarbyl linker comprising 1 to 50 carbon atoms;
wherein y is independently 0, 1, or 2; and
wherein n is independently 0, 1, 2 or 3.

In yet another aspect, the present invention provides a compound of either formula (IIIa) or formula (IIIb):

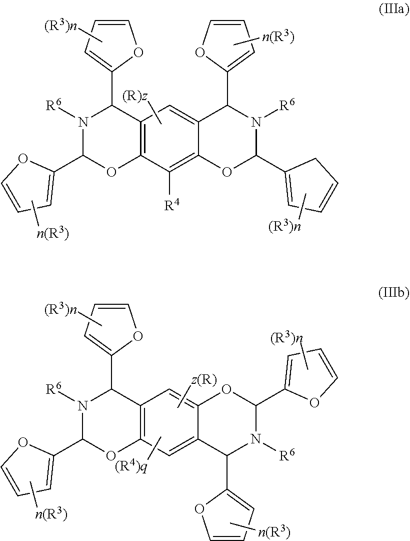

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein z is 0 or 1;
wherein q=2−z; and
wherein n is independently 0, 1, 2 or 3.

In still another aspect, the present invention provides a process for preparing a compound of formula (I) as described herein, said method comprising:
i) providing a diamine NH₂-L¹-NH₂ wherein L¹ is as described herein;
ii) adding at least one furfuraldehyde substituted with n R³ groups, and at least one phenolic compound substituted with x R groups, wherein R, R³, n and x are as described herein, with the proviso that both positions ortho to the phenolic OH group are unsubstituted on the at least one phenolic compound; and
iii) forming a compound of formula (I).

In a further aspect, the present invention provides a process for preparing a compound of formula (II), (IIIa), or (IIIb) as described herein, said method comprising:
i) providing: A) a bisphenol compound comprising a linker L² and substituted with y R groups on each benzene ring of the bisphenol compound; B) a benzene-1,4-diol compound substituted with z R groups; or C) a benzene-1,3-diol optionally substituted with 1 R group at the 5-position; wherein y, z, R and L² are as described herein;
ii) contacting the compound from step i) with at least one furfuraldehyde substituted with n R³ groups, and at least one primary amine substituted with one R⁶ group, wherein n, R³ and R⁶ are as described herein; and
iii) forming a compound of formula (II), (IIIIa), or (IIIb).

In a yet further aspect, the present invention comprises a resin composition comprising a benzoxazine compound of formula (I), (II), (IIIa), or (IIIb), as described herein.

In a still further aspect, the present invention provides the use of a benzoxazine compound of formula (I), (II), (IIIIa), or (IIIb), or a resin composition thereof, as described herein, to reinforce a material.

In another aspect, the present invention provides a method for preparing a reinforced material, said method comprising:
a) contacting a material to be reinforced with a compound of formula (I), (II), (IIIa), or (IIIb), or a resin composition thereof; and
b) allowing said compound to self-polymerise to form a reinforced material.

In another aspect, the present invention provides a reinforced material prepared, or preparable, by the methods described herein.

### BRIEF DESCRIPTION OF THE FIGURES

FIG. 1 demonstrates the viscosity vs temperature of the comparative Cardarez R111 (RTM) benzoxazine;

FIG. 2 demonstrates the viscosity development over time of comparative Cardarez R111 (RTM) benzoxazine at 100° C., 120° C. and 140° C.;

FIG. 3 demonstrates the gel-time reactivity vs temperature of comparative Cardarez R111 (RTM);

FIG. 4 demonstrates the gel-time reactivity vs temperature of Compound A;

FIG. 5 demonstrates the Tg development of thermosets cured at 180° C. vs time for a compound A thermoset and a comparative Cardarez R111 (RTM) thermoset;

FIG. 6 demonstrates the Tg development of Compound A during curing at 140° C. over 1 hour with 3%, 4% or 5% of a ferric chloride catalyst;

FIG. 7 demonstrates the Compound A viscosity build vs time at 60° C. and 100° C., with 5% ferric chloride catalyst; and

FIG. 8 demonstrates the gel times of Compound A in combination with concentrations of 1, 1.5 and 2 PHR of BCl₃ octyl dimethylamine complex at 160° C.

### DETAILED DESCRIPTION

### Definition of Terms

For the purposes of the present invention, the following terms as used herein shall, unless otherwise indicated, be understood to have the following meanings. Other terms that are not as defined below are to be understood as their normal meaning in the art.

The term “hydrocarbyl” as used herein, refers to a monovalent or divalent group, comprising hydrogen and carbon atoms, such as a major proportion (i.e., more than 50%) of hydrogen and carbon atoms, preferably consisting exclusively of hydrogen and carbon atoms. The hydrocarbyl group may be aromatic, saturated aliphatic or unsaturated aliphatic. The hydrocarbyl group may be entirely aliphatic or a combination of aliphatic and aromatic portions. In some examples, the hydrocarbyl group includes a branched aliphatic chain which is substituted by one or more aromatic groups. Examples of hydrocarbyl groups therefore include acyclic groups, as well as groups that combine one or more acyclic portions and one or more cyclic portions, which may be selected from carbocyclic, aryl and heterocyclyl groups. The hydrocarbyl group includes monovalent groups and polyvalent groups as specified and may, for example, include one or more groups selected from alkyl, alkenyl, alkynyl, carbocyclyl (e.g. cycloalkyl or cycloalkenyl), aryl and heterocyclyl. The hydrocarbyl group may contain one or more heteroatoms, such as oxygen, nitrogen, sulphur, silicon or halogen which may be part of a functional group such as an alcohol, ether, carbonyl, ester, carboxylic acid, carbonate, amide, amine, carbamate, urea, thiol, thioether, thioester, thioacid, thioamide, silane organic halide or heterocycle, the hydrocarbyl linker may contain any combination of the above insofar as it is chemically stable. Furthermore, in some embodiments, halogens may entirely replace the hydrogen component of the hydrocarbyl group (i.e. the carbon-bonded hydrogens) to give the corresponding halo-substituted analogue.

The term “alkyl” as used herein refers to a monovalent straight- or branched-chain alkyl moiety. Unless specifically indicated otherwise, the term “alkyl” does not include optional substituents. The term “haloalkyl” as used herein refers to an alkyl group substituted with one or more halogen atoms. The term “halogen” as used herein refers to any of fluorine, chlorine, bromine, or iodine.

The term “alkyloxy” as used herein refers to an alkyl group substituted with one or more hydroxy groups. The term “alkylamino” as used herein refers to an alkyl group substituted with one or more primary, secondary, or tertiary amine groups.

The term “cycloalkyl” as used herein refers to a monovalent saturated aliphatic hydrocarbyl moiety containing at least one ring, wherein said ring has at least 3 ring carbon atoms. The cycloalkyl groups mentioned herein may optionally have alkyl groups attached thereto. Examples of cycloalkyl groups include groups that are monocyclic, polycyclic (e.g., bicyclic) or bridged ring system. Examples of cycloalkyl groups include cyclopropyl, cyclobutyl, cyclopentyl, cyclohexyl and the like. The term “heterocycloalkyl” as used herein refers to a cycloalkyl group wherein the ring contains at least one heteroatom selected from oxygen, nitrogen, and sulphur. Examples of heterocycloalkyl groups include morpholine, piperidine, piperazine and the like.

The term “alkenyl” as used herein refers to a monovalent straight- or branched-chain alkyl group containing at least one carbon-carbon double bond, of either E or Z configuration unless specified. The term “alkynyl” as used herein refers to a monovalent straight- or branched-chain alkyl group containing at least one carbon-carbon triple bond. Examples of alkenyl groups include ethenyl, 2-propenyl, 1-butenyl, 2-butenyl, 3-butenyl, 1-pentenyl, 2-pentenyl, 3-pentenyl, 1-hexenyl, 2-hexenyl, 3-hexenyl and the like.

The term “aryl” as used herein refers to an aromatic carbocyclic ring system. An example of an aryl group includes a group that is a monocyclic aromatic ring system or a polycyclic ring system containing two or more rings, at least one of which is aromatic. Examples of aryl groups include aryl groups that comprise from 1 to 6 exocyclic carbon atoms in addition to ring carbon atoms. Examples of aryl groups include aryl groups that are monovalent or polyvalent as appropriate. Examples of monovalent aryl groups include phenyl, benzyl, naphthyl, fluorenyl, azulenyl, indenyl, anthryl and the like. An example of a divalent aryl group is 1,4-phenylene.

The term “heteroaryl” as used herein refers to an aromatic heterocyclic ring system wherein said ring atoms include at least one ring carbon atom and at least one ring heteroatom selected from nitrogen, oxygen and sulphur. Examples of heteroaryl groups include heteroaryl groups that are a monocyclic ring system or a polycyclic (e.g. bicyclic) ring system, containing two or more rings, at least one of which is aromatic. Examples of heteroaryl groups include those that, in addition to ring carbon atoms, comprise from 1 to 6 exocyclic carbon atoms. Examples of heteroaryl groups include those that are monovalent or polyvalent as appropriate. Examples of heteroaryl groups include pyridyl, pyrimidyl, thiopheneyl, isoxazolyl and benzo[b]furanyl groups.

The terms “furfural”, “furfuraldehyde”, “furfuryl” and “furan” are herein intended to optionally include any of the possible R³ substituents defined herein, unless explicitly stated otherwise. In the present invention, furfural is the aldehyde used in the Mannich reaction to form the benzoxazine rings. The resulting benzoxazine rings are substituted with furan at the 2- and 4-positions of the oxazine ring. Nevertheless, in the present invention a substituted furfural may be used instead of, or in combination with, unsubstituted furfural. In formulae (I), (II), (IIIa), and (IIIb) the optional furfural substituents are defined by R³.

The term “phenolic compound” as used herein refers to phenol, optionally substituted with one or more R groups at one or more of the 3-, 4-, and/or 5-positions relative to the phenol oxygen, wherein the R groups are as defined herein.

The term “bisphenol compound” as used herein refers to an organic compound comprising phenol moieties conjoined by a linker attached at any one of the 3-, 4-, or 5-positions, independently, relative to the phenol oxygen, wherein the linker is L² as defined herein. The phenol moieties are optionally substituted with one or more R groups at one or more of the 3-, 4-, and/or 5-positions relative to the phenol oxygen that are not occupied by L², wherein the R groups are as defined herein.

The present invention relates to a hitherto unknown class of difunctional benzoxazine compounds comprising two benzoxazine rings di-substituted with furfuryl groups. Two benzoxazine groups may be conjoined by a linker attached to the oxazine nitrogen atoms, or attached to the benzoxazine benzene rings, or the two benzoxazine groups may share a single benzene ring.

The difunctional benzoxazine compounds comprise at least one unsubstituted position on the benzene ring ortho to a benzoxazine oxygen atom, as this position must be unsubstituted to allow for self-polymerisation of the difunctional benzoxazine compound. As will be appreciated, “unsubstituted” herein refers to substitution by a hydrogen atom. The self-polymerisation takes place by thermal ring-opening polymerisation with or without catalyst or co-reactant. Whilst a wide range of Lewis acid catalysts (for example, including titanium isopropoxide, aluminium trichloride, Dichloro(methyl)aluminium, aluminium tribromide, iron tribromide, titanium tetrachloride, tin tetrachloride, yttrium trichloride, boron trifluoride (optionally in the form of boron trifluoride complexes, such as BF₃ etherate, BF₃ THF complex, or BF₃ amine complexes such as BF₃ methyl ethyl amine, BF₃ ethyl amine, BF₃ triethyl amine, or BF₃ dimethyl octyl amine, preferably BF₃ methyl ethyl amine), boron trichloride (optionally in the form of boron trichloride complexes, such as BCl₃ methyl ethyl amine, BCl₃ etherate, BCl₃ THF complex, or BCl₃ amine complexes such as BCl₃ ethyl amine, BCl₃ triethyl amine, or BCl₃ dimethyl octyl amine, preferably BCl₃ dimethyl octyl amine), zinc dichloride and trimethyl borane) may be used to catalyse self-polymerisation, it has been found that ferric chloride is particularly suitable for catalysing the self-polymerisation reaction. The result of the self-polymerisation is a high molecular weight thermoset polymer matrix with a high degree of cross-linkage. Resins comprising the difunctional benzoxazine compounds of the present invention are therefore useful precursors to poly-benzoxazine polymers. Advantageously, whilst the difunctional benzoxazine compounds of the present invention are liquids at standard conditions, it may not be necessary to include a solvent or diluent in such a resin. Nevertheless, solvents or diluents may be used to adjust the properties of the resin as is required.

The class of benzoxazines of the present invention may be synthesised by a Mannich reaction between a phenolic compound, a primary amine, and furfural. As would be appreciated, two equivalents of furfural are required for every one equivalent of phenolic compound and primary amine. As the present class of benzoxazines are difunctional, two equivalents of phenolic compound and two equivalents of primary amine must be reacted with four equivalents of furfural to provide one equivalent of difunctional benzoxazine. However, two equivalents of the phenolic compound or two equivalents of the primary amine may be comprised within a single difunctional molecule. For example, one equivalent of a difunctional phenolic compound or of a difunctional primary amine, may be reacted with two equivalents of a primary amine or phenolic compound, respectively, as well as four equivalents of furfural. The resulting benzoxazine will be difunctional as two benzoxazine rings are formed within the same single molecule. Therefore, the class of difunctional benzoxazine compounds of the present invention all share the feature of two benzoxazine rings, each substituted with two furan groups.

In one aspect, the benzoxazine compounds of the present invention have the general formula (I), shown below.

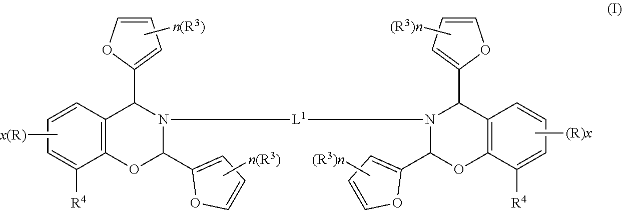

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein L¹ is a divalent hydrocarbyl linker comprising 1 to 50 carbon atoms;
wherein x is independently 0, 1, 2 or 3; and
wherein n is independently 0, 1, 2 or 3.

In some embodiments one or both benzene rings of the benzoxazines are substituted with R groups at the 3-position and/or the 5-position, relative to the oxygen of the benzoxazine ring.

In some embodiments both x values ≤2, preferably wherein both x values ≤1, and preferably wherein both x values are the same, and/or wherein both benzene rings of the benzoxazines are substituted with the same R group(s), more preferably wherein both x values=0.

Formula (I) comprises two benzoxazine moieties attached at the benzoxazine nitrogen atoms though a divalent hydrocarbyl linker group L¹. The difunctional benzoxazine may be derived from a difunctional primary amine, comprising the linker group L¹, wherein the difunctional primary amine undergoes a Mannich reaction with four equivalents of the furfural and two equivalents of a phenolic compound to form the compound of formula (I).

In some embodiments L¹ comprises 2 to 50 carbon atoms, 3 to 50 carbon atoms, 5 to 50 carbon atoms, 2 to 40 carbon atoms, 3 to 40 carbon atoms, 5 to 40 carbon atoms, 2 to 30 carbon atoms, 3 to 30 carbon atoms, 5 to 30 carbon atoms, 2 to 20 carbon atoms, 3 to 20 carbon atoms, or 5 to 20 carbon atoms.

Preferably, L¹ comprises at least one of: i) a heteroatom selected from nitrogen, oxygen, sulphur and silicon; ii) a double or triple carbon-carbon bond; or iii) an aromatic ring, heteroaromatic ring, and/or non-aromatic ring.

A list of preferred L¹ groups is provided in Table 1 below, along with their corresponding difunctional primary amines.

TABLE 1
Exemplary list diamines useful in the present invention and their
corresponding L¹ linkers.

| Diamine Name | Diamine Structure | Corresponding L¹ Structure |
|---|---|---|
| ethylene diamine | 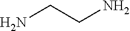 | 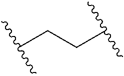 [SMILES: *CC*] |
| diethylene triamine | 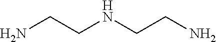 | 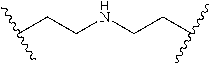 [SMILES: *CCNCC*] |
| triethylene tetramine | 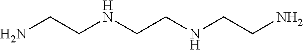 | 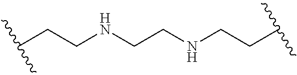 [SMILES: *CCNCCNCC*] |
| hexamethylene diamine | 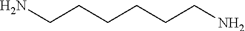 | 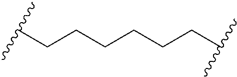 [SMILES: *CCCCCC*] |
| 2,2,4-trimethyl hexamethylene diamine | 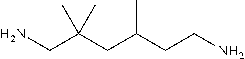 | 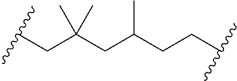 [SMILES: *CCC(C)CC(C)(C)C*] |
| 2,4,4-trimethyl hexamethylene diamine | 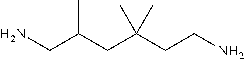 | 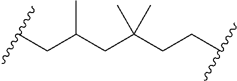 [SMILES: *CCC(C)(C)CC(C)C*] |
| Jeffamine | 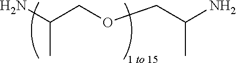 | 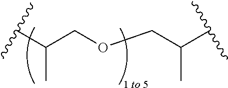 |
| metaxylylene diamine | 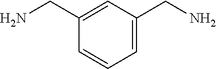 | 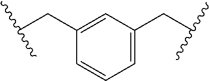 [SMILES: *Cc1cccc(C*)c1] |
| 1,3-bis(aminomethyl) cyclohexane | 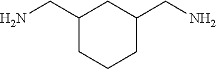 | 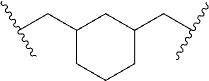 [SMILES: *CC1CCCC(C*)C1] |
| benzene-1,4-diamine | 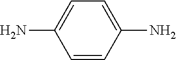 | 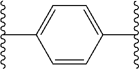 [SMILES: *c1ccc(*)cc1] |
| isophorone diamine | 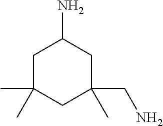 | 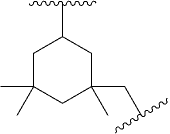 [SMILES: *CC1(C)CC(*)CC(C)(C)C1] |
| diaminocyclohexane | 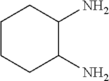 | 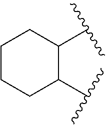 [SMILES: *C1CCCCC1*] |
| 4-Cyclohexene-1,2- diamine | 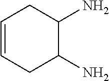 | 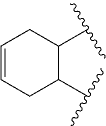 [SMILES: *C1CC=CCC1*] |
| 3-Hexyne-1,6- diamine | 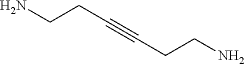 | 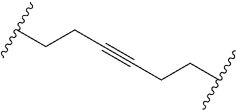 [SMILES: *CCC#CCC*] |
| bis-(p- aminocyclohexyl) methane | 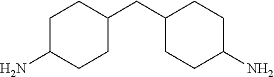 [SMILES: NC1CCC(CC2CCC(N)CC2)CC1] | 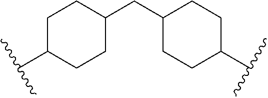 [SMILES: *C1CCC(CC2CCC(*)CC2)CC1] |
| methylene dianiline | 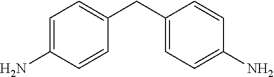 [SMILES: Nc1ccc(Cc2ccc(N)cc2)cc1] | 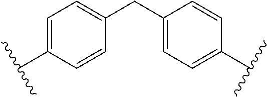 [SMILES: *c1ccc(Cc2ccc(*)cc2)cc1] |

Bis-(p-aminocyclohexyl) methane and methylene dianiline, as well as derivatives thereof are particularly preferred difunctional amines. In some embodiments, L¹ is a divalent hydrocarbyl linker comprising two rings conjoined by a divalent C₁ to C₁₀ alkyl group. The two rings may independently be carbocycles or heterocycles, but are both preferably carbocycles. The two rings may independently be 3- to 14-membered, but are preferably both 6-membered. The two rings may independently be aromatic, saturated or partially saturated, but are preferably saturated. Therefore, the two rings are most preferably cyclohexane.

In a preferred embodiment, the compound of formula (I) has the general formula (Ia), shown below.

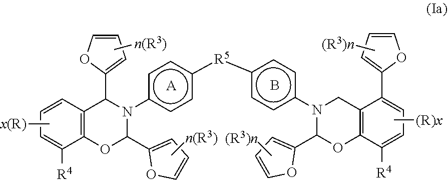

wherein ring A and ring B are independently either aromatic or saturated 6 membered carbon rings, preferably wherein rings A and B are both saturated rings (e.g. both a cyclohexane group);
wherein R⁵ is a divalent C₁ to C₁₀ alkyl group, preferably wherein R⁵ is a methylene group; and
wherein R, R³, x, and n are independently as defined hereinabove, preferably where both x values=0 and/or wherein all n values=0.

As can be seen from formula (I), substituted phenols may be used to provide the corresponding substituted benzoxazine, or unsubstituted phenols may be used to provide an unsubstituted benzoxazine. In formula (I) the optional benzoxazine substituents are defined by group R.

However, both positions ortho to the phenolic —OH group, i.e. the 2-position and 6-position, must be unsubstituted (i.e. substituted by hydrogen). This is because a first unsubstituted ortho position, is required for the Mannich reaction to take place to form the benzoxazine ring, and the second ortho position is required for the benzoxazine self-polymerisation to take place. Thus, the phenolic precursor must be 2,6-unsubstituted, in relation to the phenolic group, in order to provide a benzoxazine that is 6-unsubstituted in relation to the benzoxazine oxygen. In formula (I), the 6-unsubstituted position is defined by R⁴, wherein R⁴ is hydrogen. This leaves 3 positions available for substitution with an R group, such that 0 to 3 R groups may exist on each benzoxazine ring. The number of possible R groups on each benzoxazine ring is shown in formula (I) as x. Two identical phenolic compounds may be used to form a symmetrical difunctional benzoxazine, or two different phenolic compounds may be used to form an asymmetrical difunctional benzoxazine, as long as both phenolic compounds are 2,6-unsubstituted, in relation to the phenolic oxygen.

Preferably, naturally derived phenolic compounds may be used in order to further increase the content of bio-carbon in the resulting benzoxazine. One such example of bio derivable phenolic compound is cardanol. Cardanol is a mixture of straight chain 3-(C₁₅)alkyl and 3-(C₁₅)alkenyl phenols and is obtained from anacardic acid, the main component of cashew nutshell liquid, a by-product of cashew nut processing. Preferably cardanol is used as the phenolic compound giving a compound of formula (I) wherein one R group is present at each benzene 3-position and is selected from a —C₁₅H₃₁ straight chain alkyl group, —C₁₅H₂₉ straight chain alkenyl group, —C₁₅H₂₇ straight chain alkenyl group, and —C₁₅H₂₅ straight chain alkenyl group. As would be appreciated, a mixture of symmetrical and asymmetrical products would be obtained, all of which are within the scope of formula (I).

In another aspect, the benzoxazine compounds of the present invention have the general formula (II), shown below.

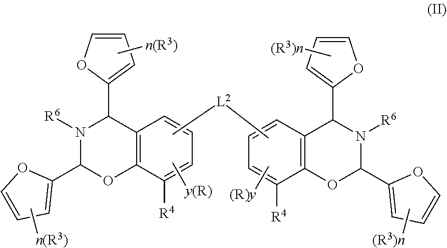

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I; wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein L² is a direct bond, a divalent group selected from: C(O), O, S, S(O), S(O)₂, or a hydrocarbyl linker comprising 1 to 50 carbon atoms;
wherein y is independently 0, 1, or 2; and
wherein n is independently 0, 1, 2 or 3.

In some embodiments both y values ≤1, and preferably wherein both y values are the same, and/or wherein both benzene rings of the benzoxazine are substituted with the same R group(s), more preferably wherein both y values=0.

Formula (II) comprises two benzoxazine moieties linked by a linker L² attached to the benzene ring of each benzoxazine. As will be appreciated, this difunctional benzoxazine may be derived from a bisphenol compound. For the purpose of the present invention, the linker L² may be independently attached to each benzene ring at any of the 3-, 4-, or 5-positions relative to the phenolic oxygen. The ortho (i.e. 2- and 6-positions) must remain unsubstituted, i.e. substituted by hydrogen in order for the Mannich reaction to proceed to form the benzoxazine ring, and for the self-polymerisation of the benzoxazine to occur.

In formula (II), L² is a direct bond, or a divalent group selected from C(O), O, S, S(O), S(O)₂ or a hydrocarbyl linker comprising 1 to 50 carbon atoms. In some embodiments L² is attached at the 4-position, relative to the benzoxazine oxygen. In some embodiments L² is selected from S(O)₂, CH₂, C(CH₃)₂, or a divalent benzene ring.

In some embodiments, L² comprises 2 to 50 carbon atoms, 3 to 50 carbon atoms, 5 to 50 carbon atoms, 2 to 40 carbon atoms, 3 to 40 carbon atoms, 5 to 40 carbon atoms, 2 to 30 carbon atoms, 3 to 30 carbon atoms, 5 to 30 carbon atoms, 2 to 20 carbon atoms, 3 to 20 carbon atoms, or 5 to 20 carbon atoms.

The linker L² may be attached to the resulting benzoxazine benzene ring at any available position, and not at the ortho position, which is occupied by R⁴ in formula (II), wherein R⁴ is hydrogen. It is preferred that L² is attached at the at the 4-position, relative to the benzoxazine oxygen. Many widely commercially available bisphenol compounds comprise linkers L² attached at both of the 4-positions relative to the phenolic oxygens, these are generally known as 4,4-bisphenols. Examples of 4,4-bisphenols suitable for use in the present invention are listed in Table 2 below.

TABLE 2
Exemplary list bisphenols useful in the present invention and their
corresponding L² linkers.

| Bisphenol compound | Bisphenol structure | L² structure |
|---|---|---|
| Bisphenol A | 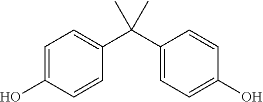 [SMILES: CC(C)(c1ccc(O)cc1)c1ccc(O)cc1] | —C(CH₃)₂— |
| Bisphenol AF | 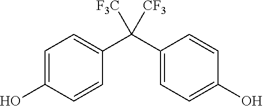 [SMILES: Oc1ccc(C(c2ccc(O)cc2)(C(F)(F)F)C(F)(F)F)cc1] | —C(CF₃)₂— |
| Bisphenol AP | 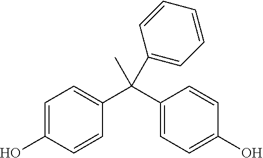 [SMILES: CC(c1ccccc1)(c1ccc(O)cc1)c1ccc(O)cc1] | —C(CH₃)Ph— |
| Bisphenol B | 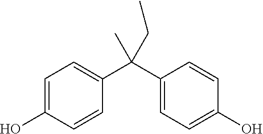 [SMILES: CCC(C)(c1ccc(O)cc1)c1ccc(O)cc1] | —C(CH₃)(CH₂CH₃)— |
| Bisphenol BP | 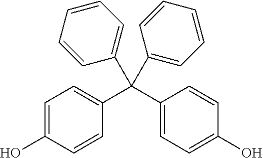 [SMILES: Oc1ccc(C(c2ccccc2)(c2ccccc2)c2ccc(O)cc2)cc1] | —CPh₂— |
| Bisphenol C | 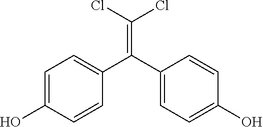 [SMILES: Oc1ccc(C(=C(Cl)Cl)c2ccc(O)cc2)cc1] | —C═CCl₂— |
| Bisphenol CP | 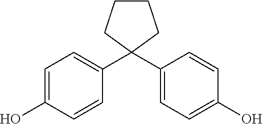 [SMILES: Oc1ccc(C2(c3ccc(O)cc3)CCCC2)cc1] | 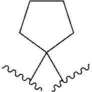 [SMILES: *C1(*)CCCC1] |
| Bisphenol E | 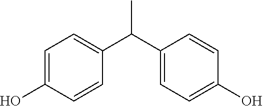 [SMILES: CC(c1ccc(O)cc1)c1ccc(O)cc1] | —C(CH₃)H— |
| Bisphenol F | 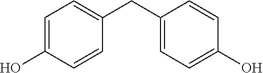 [SMILES: Oc1ccc(Cc2ccc(O)cc2)cc1] | —CH₂— |
| Bisphenol FL | 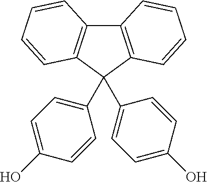 [SMILES: Oc1ccc(C2(c3ccc(O)cc3)c3ccccc3-c3ccccc32)cc1] | 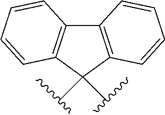 [SMILES: *C1(*)c2ccccc2-c2ccccc21] |
| Bisphenol M | 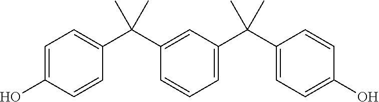 [SMILES: CC(C)(c1ccc(O)cc1)c1cccc(C(C)(C)c2ccc(O)cc2)c1] | 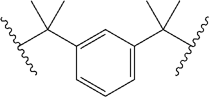 [SMILES: *C(C)(C)c1cccc(C(*)(C)C)c1] |
| Bisphenol P | 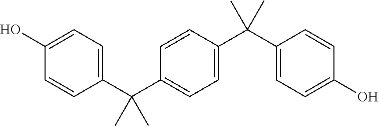 [SMILES: CC(C)(c1ccc(O)cc1)c1ccc(C(C)(C)c2ccc(O)cc2)cc1] | 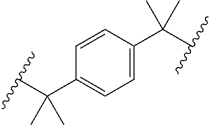 [SMILES: *C(C)(C)c1ccc(C(*)(C)C)cc1] |
| Bisphenol S | 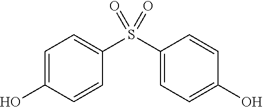 [SMILES: O=S(=O)(c1ccc(O)cc1)c1ccc(O)cc1] | —S(O)₂— |
| Bisphenol TMC | 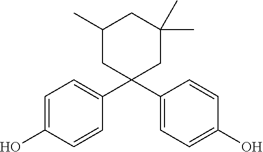 [SMILES: CC1CC(C)(C)CC(c2ccc(O)cc2)(c2ccc(O)cc2)C1] | 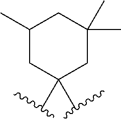 |
| Bisphenol Z | 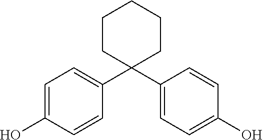 [SMILES: Oc1ccc(C2(c3ccc(O)cc3)CCCCC2)cc1] | 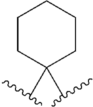 [SMILES: *C1(*)CCCCC1] |
| 4,4′-Thiodiphenol | 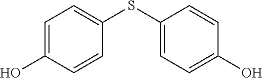 [SMILES: Oc1ccc(Sc2ccc(O)cc2)cc1] | —S— |
| 4,4′-Dihydroxydiphenyl ether | 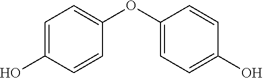 [SMILES: Oc1ccc(Oc2ccc(O)cc2)cc1] | —O— |
| 4,4′- Dihydroxybenzophenone | 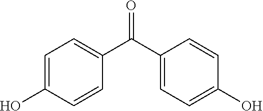 [SMILES: O=C(c1ccc(O)cc1)c1ccc(O)cc1] | —C(O)— |
| Phenolphthalein | 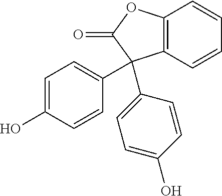 [SMILES: O=C1Oc2ccccc2C1(c1ccc(O)cc1)c1ccc(O)cc1] | 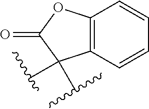 [SMILES: *C1(*)C(=O)Oc2ccccc21] |
| dicyclopentadiene bisphenol | 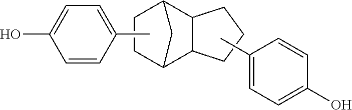 | 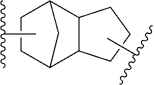 |
| 1,1′,4′,1″-Terphenyl- 4,4″-diol | 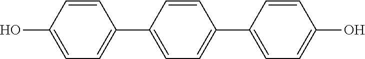 [SMILES: Oc1ccc(-c2ccc(-c3ccc(O)cc3)cc2)cc1] | 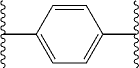 [SMILES: *c1ccc(*)cc1] |
| 1,4-Bis(4- hydroxybenzyl)benzol | 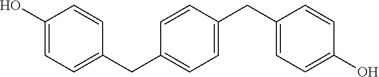 [SMILES: Oc1ccc(Cc2ccc(Cc3ccc(O)cc3)cc2)cc1] | 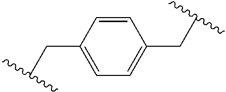 [SMILES: *Cc1ccc(C*)cc1] |
| 4,4′-Biphenol | 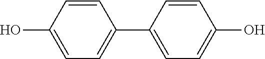 [SMILES: Oc1ccc(-c2ccc(O)cc2)cc1] | Direct bond |

As would be appreciated, the L² groups would be conserved during a Mannich reaction and would thus be present in the compound of formula (II) when derived from the corresponding bisphenol compound precursor. Whilst the bisphenol compounds listed in Table 2 are all 4,4-bisphenols, the scope of the invention also includes any isomers of these bisphenol compounds wherein the L² linker is attached at any combination of the 3-, 4- and 5-positions.

This leaves two positions available for substitution with an R group, such that 0 to 2 R groups may exist on each benzoxazine ring. The number of possible R groups on each benzoxazine ring is shown in formula (II) as y. A symmetrical bisphenol compound, with the same or no substituents on each phenol group may be used to form a symmetrical difunctional benzoxazine, or an asymmetrical bisphenol compound, with differing substituents on each phenol group may be used may be used to form an asymmetrical difunctional benzoxazine, as long as both phenol groups are 2,6-unsubstituted, in relation to the phenolic oxygen, in both cases the total number of R groups is y+y.

In another aspect, the benzoxazine compounds of the present invention have the general formula (IIIIa), shown below.

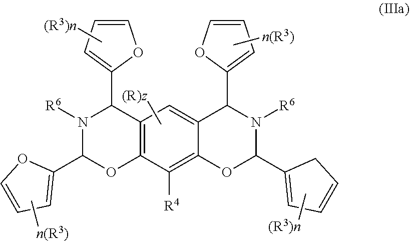

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to Cr alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein z is 0 or 1; and
wherein n is independently 0, 1, 2 or 3.

In this aspect, the difunctional benzoxazine may be derived from a 1,3-benzene-diol. Two oxazine rings are formed on the shared benzene ring, giving a difunctional benzoxazine with a shared benzene ring. The compound of formula (IIIIa) may or may not be substituted with an R group at the 5-position of the benzene ring relative to the benzoxazine oxygens which are considered to occupy the 1- and 3-positions. The corresponding 4- and 6-positions of the benzene ring are occupied carbon atoms that form part of the benzoxazine ring. The compound of formula (IIIa) must be unsubstituted at the 2-position, relative to the benzoxazine oxygens which are considered to occupy the 1- and 3-positions, so that self-polymerisation of the resulting benzoxazine may take place at this position. The 2-position is occupied by R⁴ in formula (IIIIa), wherein R⁴ is hydrogen. Therefore, the 1,3-benzene-diol precursor must be unsubstituted at the 4- and 6-positions in order to allow the oxazine rings to be formed at these positions, and the 2-position must be unsubstituted to allow for self-polymerisation of the resulting benzoxazine compound.

This leaves only one position available for optional substitution on the benzoxazine ring. The number of possible R groups on the benzoxazine ring is shown in formula (IIIIa) as z. Thus, z is either 1 or 0. In a preferred embodiment of the compound of formula (IIIIa), z=0 and so the compound of formula (IIIIa) is unsubstituted at the 5-position. The corresponding precursor without substitution at the 5-position, i.e. 1,3-benzene-diol, is known as resorcinol.

In another embodiment, the compound of formula (IIIa) is substituted with a C₁ to C₂₀ alkyl group or a C₂ to C₂₀ alkenyl group at the 5-position. The corresponding precursor 1,3-benzene-diols, i.e. 5-alkylresorcinols and 5-alkeneylresorcinols are readily available from natural sources, one such example is 5-Pentadecenylresorcinol, also known as bilobol, which is found in Ginkgo biloba fruits.

In a related aspect, the benzoxazine compounds of the present invention have the general formula (IIIb), shown below.

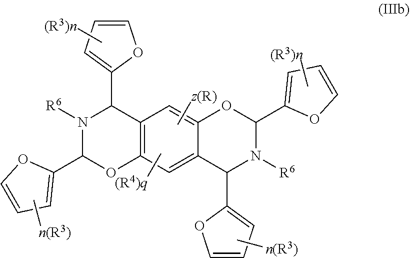

wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein z is 0 or 1;
wherein q=2−z; and
wherein n is independently 0, 1, 2 or 3.

In this aspect, the difunctional benzoxazine may be derived from a 1,4-benzene-diol. Two oxazine rings are formed on the shared benzene ring, giving a difunctional benzoxazine with a shared benzene ring. The compound of formula (IIIb) may or may not be substituted with an R group at one of the two positions on the benzene ring ortho to the benzoxazine oxygens that is not occupied by a carbon atom of the benzoxazine ring. At least one of, preferably both of, the positions on the benzene ring ortho to the benzoxazine oxygens that is not occupied by a carbon atom of the benzoxazine ring, must remain unsubstituted so that self-polymerisation of the benzoxazine of formula (IIIb) may take place.

At least three positions on the 1,4-benzene-diol precursor must be unsubstituted, two of which must be unsubstituted in order to allow the oxazine rings to be formed at these positions, and a further one, preferably two must be, so that self-polymerisation of the resulting benzoxazine may take place.

The number of possible R groups on the benzoxazine ring is shown in formula (IIIb) as z. z is either 1 or 0. If one position ortho to a benzoxazine oxygen that is not occupied by a carbon atom of the benzoxazine ring is substituted with an R group, then the other position ortho to the other benzoxazine oxygen that is not occupied by a carbon atom of the other benzoxazine ring must be substituted with R⁴, wherein R⁴ is hydrogen. If neither of the positions ortho to the benzoxazine oxygens that are not occupied by carbon atoms of the benzoxazine rings are substituted with an R group, then both of these positions must be substituted with R⁴, wherein R⁴ is hydrogen. Therefore, the number of R⁴ groups on the benzoxazine ring is shown in formula (IIIb) is q, wherein q=2−z.

In some embodiments, the compounds of any one of Formulae (I), (II), (IIIa) and (IIIb) are selected from those wherein R is independently selected from: C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —O(CO)H, —O(CO)R¹, —NH(CO)H, —NH(CO)R¹, —NR¹(CO)H, —NR¹(CO)R¹, —SH, —SR¹, —SiR¹₃, —F, —Cl, —Br, —I, C₆ to C₁₄ aryl optionally substituted with one or more R² group, and C₂ to C₉ heteroaryl optionally substituted with one or more R² group;
wherein R¹ is independently selected from: C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀, heterocycloalkyl, C₁ to C₂₀ alkoxy, C₁ to C₂₀ alkylamino, C₆ to C₁₄ aryl, and C₂ to C₉ heteroaryl; and wherein R² is independently selected from: C₁ to C₂₀ alkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —F, —Cl, —Br, or —I.

In some embodiments, the compounds of any one of Formulae (I), (II), (IIIa) and (IIIb) are selected from those wherein R is independently selected from: C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, —OH, —OR¹, —NH₂, —NHR¹, —NR¹₂, C₆ to C₁₄ aryl optionally substituted with one or more R² group, or C₂ to C₉ heteroaryl optionally substituted with one or more R² group; preferably —OH;
wherein R¹ is independently selected from: C₁ to C₂₀ alkyl, C₃ to C₂₀ cycloalkyl or C₂ to C₂₀ alkenyl;
wherein R² is independently selected from: C₁ to C₂₀ alkyl, —OH, —NH₂, —OC₁ to C₂₀ alkyl, —NHC₁ to C₂₀ alkyl or —N(C₁ to C₂₀ alkyl)₂, preferably —OH.

In some embodiments, the compounds of any one of Formulae (I), (II), (IIIa) and (IIIb) are selected from those wherein R is independently selected from: C₁ to C₁₀ alkyl, C₁ to C₁₀ haloalkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyl-C₆ to C₁₄ aryl, C₁ to C₁₀ alkyl-C₂ to C₉ heteroaryl, C₁ to C₁₀ alkyl-C₃ to C₁₀ cycloalkyl, C₁ to C₂₀ alkyl-C₃ to C₁₀ heterocycloalkyl, C₁ to C₁₀ alkyloxy, C₁ to C₁₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₁₀ alkyl, C₁ to C₁₀ haloalkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyl-C₆ to C₁₄ aryl, C₁ to C₁₀ alkyl-C₂ to C₉ heteroaryl, C₁ to C₁₀ alkyl-C₃ to C₁₀ cycloalkyl, C₁ to C₁₀ alkyl-C₃ to C₁₀ heterocycloalkyl, C₁ to C₁₀ alkoxy, C₁ to C₁₀ alkylamino, C₆ to C₁₄ aryl, and C₂ to C₉ heteroaryl;
wherein R² is independently selected from: C₁ to C₁₀ alkyl, C₁ to C₁₀ haloalkyl, C₃ to C₁ cycloalkyl, C₂ to C₁₀ alkenyl, C₂ to C₁₀ alkynyl, —OH, —NH₂, —OC₁ to C₁₀ alkyl, —NHC₁ to C₁₀ alkyl, —N(C₁ to C₁₀ (alkyl)₂, —NO₂, —CN, —F, —Cl, —Br, and —I.

In some embodiments, the compounds of any one of Formulae (I), (II), (IIIIa) and (IIIb) are selected from those wherein R is independently selected from: C₁ to C₁₀ alkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyl-C₆ to C₁₄ aryl, C₁ to C₁₀ alkyl-C₂ to C₉ heteroaryl, C₁ to C₂₀ alkyl-C₃ to C₁₀ cycloalkyl, C₁ to C₁₀ alkyl-C₃ to C₁₀ heterocycloalkyl, C₁ to C₁₀ alkyloxy, C₁ to C₁₀ alkylamino, —OH, —OR¹, —NH₂, —NHR¹, —NR¹₂, —O(CO)H, —O(CO)R¹, —NH(CO)H, —NH(CO)R¹, —NR¹(CO)H, —NR¹(CO)R¹, —SH, —SR¹, —SiR¹₃, —F, —Cl, —Br, —I, C₆ to C₁₄ aryl optionally substituted with one or more R² group, and C₂ to C₉ heteroaryl optionally substituted with one or more R² group;
wherein R¹ is independently selected from: C₁ to C₁₀ alkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyl-C₆ to C₁₄ aryl, C₁ to C₁₀ alkyl-C₂ to C₉ heteroaryl, C₁ to C₁₀ alkyl-C₃ to C₁₀ cycloalkyl, C₁ to C₁₀ alkyl-C₃ to C₁₀, heterocycloalkyl, C₁ to C₁₀ alkoxy, C₁ to C₁₀ alkylamino, C₆ to C₁₄ aryl, and C₂ to C₉ heteroaryl; and
wherein R² is independently selected from: C₁ to C₁₀ alkyl, C₃ to C₁₀ cycloalkyl, C₂ to C₁₀ alkenyl, C₂ to C₁₀ alkynyl, —OH, —NH₂, —OC₁ to C₁₀ alkyl, —NHC₁ to C₁₀ alkyl, —N(C₁ to C₁₀ alkyl)₂, —F, —Cl, —Br, or —I.

In some embodiments, the compounds of any one of Formulae (I), (II), (IIIa) and (IIIb) are selected from those wherein R is independently selected from: C₁ to C₁₀ alkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyloxy, C₁ to C₁₀ alkylamino, —OH, —OR¹, —NH₂, —NHR¹, —NR¹₂, C₆ to C₁₄ aryl optionally substituted with one or more R² group, or C₂ to C₉ heteroaryl optionally substituted with one or more R² group; preferably —OH;
wherein R¹ is independently selected from: C₁ to C₁₀ alkyl, C₃ to C₁₀ cycloalkyl or C₂ to C₁₀ alkenyl;
wherein R² is independently selected from: C₁ to C₁₀ alkyl, —OH, —NH₂, —OC₁ to C₁₀ alkyl, —NHC₁ to C₁₀ alkyl or —N(C₁ to C₁₀ alkyl)₂, preferably —OH.

It is preferred that the R group(s) present on the benzoxazine ring(s) of compounds of any of Formulae (I), (II), (IIIa), and/or (IIIb) are activating groups. The activating properties of an activating group derive from their ability to donate electrons into an aromatic system, thus enhancing its affinity towards electrophiles, i.e. activating groups are electron donating groups. The degree of electron donation of an electron donating group is proportional to its strength as an activating group. These properties can be determined in relation to a specific substituent using the Hammett equation:

σ=log Kₓ−log K_{H}

K_{H} is the ionisation constant for benzoic acid in water at 25° C. and Kₓ is the ionisation constant in water at 25° C. for a benzoic acid substituted with the electron donating group in question. σ is the Hammett substituent constant, the more negative the Hammett substituent constant is, the more electron donating the substituent is, and therefore the stronger an activating group the substituent is. The Hammett substituent constant can thus be used to measure the strength of an activating group.

The Hammett substituent constant of a substituent will differ depending on its position on the benzoic acid (e.g. -para, -meta or -ortho), the -para, -meta and -ortho Hammett substituent constants thus represent the activating effect the substituent has on the position -para, -meta or -ortho to the substituent, respectively. Hammett substituent constants for substituents at the -ortho position cannot be accurately experimentally calculated due to the interference of steric effects. However, -ortho Hammett substituent constants can be estimated using density functional theory and are found to closely correspond to the -para Hammett substituent constants. Hammett substituent constants of an electron donating group at the -meta position tend to be significantly smaller than those at the -para and -ortho positions, which gives rise to the preferentially -ortho and -para regioselective directing effects of activating groups. -Meta Hammett substituent constants are thus of little relevance to electrophilic aromatic substitution.

The Hammett substituent constant (a) herein refers to the Hammett substituent constant of the substituent in question at the -para position, which is approximately equivalent to the Hammett substituent constant at the -ortho position and corresponds to the increase in reactivity of the aromatic ring with regard to electrophilic aromatic substitution at both the -para and -ortho positions. As would be appreciated, a more negative Hammett substituent constant corresponds to an increase in reactivity.

Literature values of Hammett substituent constants for common substituents would be known to the skilled person and can be found by reference to textbooks, for example, Hansch. C, Leo. A, Substituent constants for correlation analysis in chemistry and biology, New York NY: Wiley, 1979. Hammett substituent constants could also be easily measured by the skilled person by methods known in the art, see Hammett. P, The Effect of Structure upon the Reactions of Organic Compounds. Benzene Derivatives, J. Am. Chem. Soc., 1937, 59, 1, 96-103 and Hansch. C et al A Survey of Hammett Substituent Constants and Resonance and Field Parameters, Chem. Rev., 1991, 91, 165-195.

Table 3 provides examples of Hammett substituent constants, as reported by Hammett. P, The Effect of Structure upon the Reactions of Organic Compounds. Benzene Derivatives, J. Am. Chem. Soc., 1937, 59, 1, 96-103 and Hansch. C et al A Survey of Hammett Substituent Constants and Resonance and Field Parameters, Chem. Rev., 1991, 91, 165-195, as well as Hansch. C, Leo. A, Substituent constants for correlation analysis in chemistry and biology, New York NY: Wiley, 1979. As described hereinabove, Hammett Substituent constants reported in Table 3 are the para Hammett Substituent Constants.

TABLE 3
Hammett substituent constants

| | | Hammett Substituent |
|---|---|---|
| Group | Structure | Constant (σ) |
| Dimethyl amine | —NMe₂ | −0.830 |
| Primary amine | —NH₂ | −0.660 |
| Hydroxy | —OH | −0.370 |
| Methoxy | —OMe | −0.268 |
| Ethoxy | —OEt | −0.250 |
| Methyl | —CH₃ | −0.170 |
| Benzyl | —CH₂C₆H₅ | −0.090 |
| Trimethyl silyl | —SiMe₃ | −0.070 |
| Phenyl | —C₆H₆ | −0.010 |

In some embodiments, the compounds of any one of Formulae (I), (II), (IIIIa) and (IIIb) are selected from those wherein R is selected from a substituent having a Hammett substituent constant (a) of less than zero, more preferably less than −0.050, even more preferably less than −0.100, most preferably less than −0.200.

Unsubstituted furfural is a particularly desirable feedstock due to its low toxicity and abundant availability from natural sources. Related furan 2-carbaldehydes are also derivable from natural sources and thus also desirable chemical feedstocks, in particular 5-(Hydroxymethyl)-2-furaldehyde, 5-(methoxymethyl)-2-furaldehyde, and 5-methy-2-furfuraldehyde are preferred furfuraldehydes useful in the present invention. Unsubstituted furfural is the most preferred.

In some embodiments, the compounds of any one of Formulae (I), (II), (IIIIa) and (IIIb) are selected from those wherein R³ is independently selected from: C₁ to C₆ alkyl, C₁ to C₆ alkoxy, C₁ to C₆ alkyloxy, and —OH, preferably —CH₃, —CH₂OH, or —CH₂OCH₃.

In some embodiments, the compounds of any one of Formulae (I), (II), (IIIIa) and (IIIb) are selected from those wherein all n values ≤1, and preferably wherein all n values are the same, and/or wherein all R³ groups are the same, and/or wherein all R³ groups are attached at the position ortho to the furan oxygen; more preferably wherein all n values=0.

In some embodiments, each R⁶ group of compounds (II), (IIIa), and/or (IIIb) is independently selected from: —H, —OH, C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, C₆ to C₁₄ aryl, and C₂ to C₉ heteroaryl; wherein R⁶ is optionally substituted with one or more R⁷ groups; wherein R⁷ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl, and C₂ to C₉ heteroaryl.

In some embodiments, the compounds of any one of Formulae (II), (IIIa) and (IIIb) are selected from those wherein R⁶ is independently selected from: —H, —OH, C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, C₆ to C₁₄ aryl, and C₂ to C₉ heteroaryl; wherein R⁶ is optionally substituted with one or more R⁷ groups; wherein R⁷ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl, and C₂ to C₉ heteroaryl.

In some embodiments, the compounds of any one of Formulae (II), (IIIa) and (IIIb) are selected from those wherein R⁶ is independently selected from: H, —OH, C₁ to C₁₀ alkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyloxy, C₁ to C₁₀ alkylamino, C₆ to C₁₄ aryl, and C₂ to C₉ heteroaryl; wherein R⁶ is optionally substituted with one or more R⁷ groups; wherein R⁷ is independently selected from: C₁ to C₁₀ alkyl, C₁ to C₁₀ haloalkyl, C₃ to C₁ cycloalkyl, C₃ to C₁₀ heterocycloalkyl, C₂ to C₁₀ alkenyl, C₂ to C₁₀ alkynyl, C₁ to C₁₀ alkyloxy, C₁ to C₁₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl, and C₂ to C₉ heteroaryl.

In some embodiments, the compounds of any one of Formulae (II), (IIIa) and (IIIb) are selected from those wherein R⁶ is independently selected from: H, —OH, C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₆ to C₁₄ aryl, and C₂ to C₉ heteroaryl; wherein R⁶ is optionally substituted with one or more R⁷ groups; wherein R⁷ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, —OH, —OR¹, —NR¹₂, —C(O)OR¹, —C(O)NH₂, —C(O)NHR¹, —C(O)NR¹₂, —O(CO)R¹, —NH(CO)H, —NH(CO)R¹, —NR¹(CO)H, —NR¹(CO)R¹, —SH, —SR¹, —SiR¹₃, —NO₂, —CN, —F, —Cl, —Br, —I, C₆ to C₁₄ aryl, and C₂ to C₉ heteroaryl.

In some embodiments, the compounds of any one of Formulae (II), (IIIa) and (IIIb) are selected from those wherein R⁶ is independently selected from: —H, —OH, C₁ to C₁₀ alkyl, C₃ to C₁₀ cycloalkyl, C₃ to C₁₀ heterocycloalkyl, C₂ to C₁₀ alkenyl, C₂ to C₁₀ alkynyl, C₁ to C₁₀ alkyloxy, C₆ to C₁₄ aryl, and C₂ to C₉ heteroaryl; wherein R⁶ is optionally substituted with one or more R⁷ groups; wherein R⁷ is independently selected from: C₁ to C₁₀ alkyl, C₁ to C₁₀ haloalkyl, C₃ to C₁₀ cycloalkyl, C₃ to C₁₀ heterocycloalkyl, C₂ to C₁₀ alkenyl, C₂ to C₁ alkynyl, C to C₁₀ alkyloxy, —OH, —OR¹, —NR¹₂, —C(O)OR¹, —C(O)NH₂, —C(O)NHR¹, —C(O)NR¹₂, —O(CO)R¹, —NH(CO)H, —NH(CO)R¹, —NR¹(CO)H, —NR¹(CO)R¹, —SH, —SR¹, —SiR¹₃, —NO₂, —CN, —F, —Cl, —Br, —I, C₆ to C₁₄ aryl, and C₂ to C₉ heteroaryl.

In some embodiments, the compounds of any one of Formulae (II), (IIIa) and (IIIb) are selected from those wherein R⁶ is independently selected from: H, —OH, C₁ to C₂₀ alkyl, C₆ to C₁₄ aryl, and C₂ to C₉ heteroaryl; wherein R⁶ is optionally substituted with one or more R⁷ groups; wherein R⁷ is independently selected from: C₁ to C₂₀ alkyl, —OH, —OR¹, —NR¹₂, —NO₂, —CN, —F, —Cl, —Br, and —I.

In some embodiments, the compounds of any one of Formulae (II), (IIIa) and (IIIb) are selected from those wherein R⁶ is independently selected from: —H, C₁ to C₁₀ alkyl, C₆ to C₁₄ aryl, and C₂ to C₉ heteroaryl; wherein R⁶ is optionally substituted with one or more R⁷ groups; wherein R⁷ is independently selected from: C₁ to C₁₀ alkyl, —OH, —OR¹, —NR¹₂, —NO₂, —CN, —F, —Cl, —Br, and —I. In preferred embodiments, the compounds of any one of Formulae (II), (IIIa) and (IIIb) are selected from those wherein, R⁶ is a phenyl group.

In some embodiments, the compounds of any one of Formulae (II), (IIIa) and (IIIb) are selected from those wherein, both R⁶ groups are the same. In other embodiments, the compounds of any one of Formulae (II), (IIIa) and (IIIb) are selected from those wherein, both R⁶ groups are the different.

The present invention also provides processes for synthesising the compounds of formulae (I), (II), (IIIa) and (IIIb).

In one aspect the present invention also provides a process for preparing a compound of formula (I) comprising the steps of:
i) providing a diamine NH₂-L¹-NH₂ wherein L¹ is as defined herein;
ii) adding at least one furfuraldehyde substituted with n R³ groups, and at least one phenolic compound substituted with x R groups, wherein R, R³, n and x are as defined herein, with the proviso that both positions ortho to the phenolic OH group are unsubstituted on the at least one phenolic compound; and
iii) forming a compound of formula (I).

Whilst the exact ratio of diamine to phenolic compound to furfuraldehyde used in this process is 1:2:4, differing molar ratios of these precursors may be utilised in some embodiments. Optionally, the molar ratio of the diamine to the at least one furfuraldehyde is from 1:2 to 1:20, preferably from 1:3 to 1:10, more preferably from 1:4 to 1:8; and/or wherein the molar ratio of the diamine to the at least one phenolic compound is from 1:1 to 1:5, preferably from 1:1.5 to 1:4, more preferably from 1:2 to 1:3.

There exists a wide range of orders of addition that fall within the scope of the processes for synthesising the compound of formula (I). Different orders of addition of the reactants may be used to favour specific products.

In one embodiment the diamine is contacted with the at least one furfuraldehyde prior to the addition of the at least one phenolic compound.

In one embodiment the diamine is contacted with a portion of the at least one furfuraldehyde prior to the addition of the at least one phenolic compound, wherein the remaining furfuraldehyde is added after and/or simultaneously to addition of the at least one phenolic compound.

In one embodiment the diamine is contacted with the at least one phenolic compound and the at least one furfuraldehyde simultaneously.

In one embodiment, two different phenolic compounds are added separately. This embodiment is suited to the preparation of asymmetrical benzoxazines.

In one embodiment, only one phenolic compound is added. This embodiment is suited to the preparation of symmetrical benzoxazines.

In another aspect, the present invention provides a process for preparing a compound of formula (II), (IIIIa) or (IIIb) comprising the steps of:
i) providing A) a bisphenol compound comprising a linker L² and substituted with y R groups on each benzene ring of the bisphenol compound; B) a benzene-1,4-diol compound substituted with z R groups; or C) a benzene-1,3-diol optionally substituted with 1 R group at the 5-position; wherein y, z, R and L² are as defined herein;
ii) contacting the compound from step i) with at least one furfuraldehyde substituted with n R³ groups, and at least one primary amine substituted with one R⁶ group, wherein n, R³ and R⁶ are as defined herein; and
iii) forming a compound of formula (II), (IIIIa), or (IIIb).

As described above, this process yields a compound of formula (II) when a bisphenol compound precursor is used, a compound of formula (IIIIa) when a 1,3-benzene-diol is used, or a compound of formula (IIIb) when a 1,4-benzene-diol is used. In the aspects relating to formula (II), (IIIIa) and (IIIb), two equivalents of primary amine are used in the preparation of the benzoxazine compound, the primary amine has one substituent defined herein as R⁶. The primary amine becomes incorporated into the benzoxazine ring to provide the benzoxazine nitrogen substituted with R⁶. A single primary amine may be used so that both R⁶ groups are the same, or multiple primary amines may be used so that both R⁶ groups are different from each other.

Whilst the exact ratio of phenolic compound or benzene-diol, to primary amine to furfuraldehyde used in this process is 1:2:4, differing molar ratios of these precursors may be utilised in some embodiments. Optionally, the molar ratio of the bisphenol compound or benzenediol to the at least one furfuraldehyde is from 1:2 to 1:20, preferably from 1:3 to 1:10, more preferably from 1:4 to 1:8; and/or wherein the molar ratio of the bisphenol compound or benzenediol to the at least one primary amine is from 1:1 to 1:5, preferably from 1:1.5 to 1:4, more preferably from 1:2 to 1:3.

There exists a wide range of orders of addition that fall within the scope of the processes for synthesising the compounds of formulae (II), (IIIa) and (IIIb). Different orders of addition of the reactants may be used to favour specific products.

In one embodiment the at least one primary amine, is contacted with the at least one furfuraldehyde prior to the addition of the bisphenol compound or benzenediol.

In one embodiment the at least one primary amine, is contacted with a portion of the at least one furfuraldehyde prior to the addition of the bisphenol compound or benzenediol, wherein the remaining furfuraldehyde is added after and/or simultaneously to addition of the bisphenol compound or benzenediol, respectively.

In one embodiment the at least one primary amine, is contacted with the bisphenol compound or benzenediol, and the at least one furfuraldehyde simultaneously.

In one embodiment two different primary amines are added separately. This embodiment is suited to the preparation of asymmetrical benzoxazines.

In one embodiment only one primary amine is added. This embodiment is suited to the preparation of symmetrical benzoxazines.

The compounds of the present invention may be prepared over a range of different temperatures that the skilled person would be familiar with from conventional Mannich chemistry and conventional benzoxazine preparations. The compounds of the present invention may, for instance, be prepared at room temperature and the process may be accelerated by heating and/or the use of a catalyst. Preferably, the temperature of the reaction is between 25° C. and 150° C., more preferably the temperature of the reaction is between 40° C. and 100° C. The reaction may optionally be catalysed wherein either: the catalyst is i) an acid catalyst, for example an acid catalyst selected from HCl, trifluoroacetic acid, methane sulphonic acid, p-toluenesulphonic acid, trifluoromethanesulphonic acid, benzoic acid and mixtures thereof; or ii) a basic catalyst, for example a basic catalyst selected from NaOH, Na₂CO₃, triethylamine, triethanolamine and mixtures thereof. A broad range of organic solvents are suitable for use in the reaction, for example MeCN, benzene, methanol, ethanol, IPA, butanol, chloroform, DCM, diethyl ether, DMF, dioxane, ethyl acetate, petroleum ether, kerosine, pentane, hexane, heptane, MTBE, NMP, THF, toluene, xylene and mixtures thereof. Toluene is the most preferred solvent for use in the reaction.

As would be appreciated, the Mannich reaction and benzoxazine cyclisation reaction produce water. The skilled person is capable of employing methods of removing water produced during the reaction along with any solvent used, in order to yield the final product, such as boiling, distillation, vacuum distillation, azeotroping, dean stark, precipitation etc.

In a yet further aspect, the present invention comprises a resin composition comprising a benzoxazine compound of formula (I), (II), (IIIIa), or (IIIb), as described herein. Any suitable organic solvent may be used as a solvent or diluent in a resin composition of the present invention, for example MeCN, benzene, methanol, ethanol, IPA, butanol, chloroform, DCM, diethyl ether, DMF, dioxane, ethyl acetate, petroleum ether, kerosine, pentane, hexane, heptane, MTBE, NMP, THF, toluene, xylene and mixtures thereof. Toluene is a particularly suitable solvent or diluent for difunctional benzoxazine compounds and resins thereof of the present invention. As would be appreciated, methods of forming and handling a resin composition from a given monomeric compound would be known by the skilled person.

### Reinforced Materials

The benzoxazine compounds of the present invention, or resins thereof are particularly useful in the manufacture of reinforced materials. In particular, thermoset polymer matrixes, such as fibre-reinforced plastic, comprising fibrous materials, for example aramid fibre, basalt, wood fibre, glass fibre, carbon fibre or flax. The material to be reinforced is contacted with a difunctional benzoxazine compound of the present invention, or resin thereof, to form a pre-preg, followed by curing of the difunctional benzoxazine compound by self-polymerisation to form cross-linkages, to thereby reinforce the material. A coating may be formed on a material by contacting the surface of the material with a difunctional benzoxazine compound of the present invention, or resin thereof, followed by allowing self-polymerisation of the difunctional benzoxazine compound. Self-polymerisation of the difunctional benzoxazine compound can occur at ambient temperature or elevated temperature, with or without a catalyst or co-reactant.

Self-polymerisation of the difunctional benzoxazine also allows the compounds of the invention to be used for the purpose of liquid moulding, film adhesives, composite construction, material bonding and repair, as in the case of conventional benzoxazines. As the skilled person will appreciate from the present disclosure, the properties of the difunctional benzoxazine compounds also find particular utility in resin formation, resin polymerisation, and formation of reinforced materials such as fibre-reinforced plastic.

The polymer formed by self-polymerisation of the difunctional benzoxazine compounds of the present invention has exposed phenol groups, due to the benzoxazine ring opening reaction. This means that further cross-linkage or curing can be achieved using epoxy resin, the epoxide groups of which are capable of reacting with the exposed phenol groups. An epoxy-based resin may be applied to, or incorporated into, a difunctional benzoxazine compound of the present invention, or resin composition thereof, or a pre-preg or reinforced material formed therefrom, in order to provide further curing, hardening, cross-linkage and reinforcement. Hybridisation with epoxy-based resins may be used to increase the Tg of a benzoxazine thermoset, or material comprising or reinforced with the same.

Examples of epoxy-based resins suitable for use in the present invention include polyglycidyl ethers of polyhydric phenols, epoxidised novolacs or similar glycidated polyphenolic resins, glycidated bisphenols, such as glycidated bisphenol A or F, or halogenated (e.g. chlorinated or fluorinated) analogues thereof, polyglycidyl ethers of alcohols, glycols or polyglycols, and polyglycidyl esters of polycarboxylic acids. Preferred examples of epoxy resins are polyglycidyl ethers of a polyhydric phenol. Polyglycidyl ethers of polyhydric phenols can be produced, for example, by reacting an epihalohydrin with a polyhydric phenol in the presence of an alkali. Examples of suitable polyhydric phenols include: 2,2-bis (4-hydroxyphenyl) propane (bisphenol-A); 2,2-bis(4-hydroxy-3-tert-butylphenyl)propane; 1,1-bis(4-hydroxyphenyl) ethane; 1,1-bis(4-hydroxyphenyl) isobutane; bis(2-hydroxy-1-naphthyl) methane; 1,5-dihydroxynaphthalene; 1,1-bis(4-hydroxy-3-alkylphenyl) ethane and the like. Commercial examples of preferred epoxy resins that may be used include EPILOK 60-600.

The reinforced material may be prepared by applying a layer of a resin as defined herein, onto a bottom film carrier layer. Glass fibres, carbon fibres, flax, or any other fibrous material as the reinforcement may then then be applied to the upper surface of the resin on the film carrier. A further layer of the resin is applied to sandwich the fibrous material between the layers. A top film may be applied to the upper layer. The resulting layered composition may subsequently be compressed as part of forming the reinforced material.

As will be appreciated, in some embodiments, the reinforced material of the present invention may be formed from a sheet-molding compound (SMC) comprising a compound according to formula (I), (II), (IIIa), or (IIIb), or resin thereof, and fibrous material as defined above. Methods of preparing such SMC materials are known in the art and can be readily adapted for use in preparing reinforced materials as defined herein. The SMC material may further include additives selected from hardeners, accelerators, fillers, pigments, fire retardants and/or any other components, as required. For example, the sheet-form polymeric material may also include melamine, which is useful as a fire retardant. The SMC may include a further thermoplastic material.

In some embodiments, the reinforced material of the present invention may comprise a number of layers, the layers may be joined together in a variety of ways. For instance, where air-tight sealing coating material comprising an elastomer is used, the same coating material may be used to bond the first insulating layer to one or more adjacent layers of the layered composite material panel. Alternatively, or in addition, SMC material comprising a compound according to formula (I), (II), (IIIIa), or (IIIb) may be bonded to one or more adjacent layers during curing of the reinforced material, for instance using heat and/or pressure. In addition, a variety of known adhesives may be used to bond the individual layers of the layered composite material panel together. Preferably, pressure is applied to the layered composite material during the bonding step so as to ensure good adhesion of the layers. As noted above, where one or more layers comprises a compound according to formula (I), (II), (IIIIa), or (IIIb), or resin thereof, (e.g. as part of an SMC material) the application of pressure may also assist in the curing of the compound according to formula (I), (II), (IIIIa), or (IIIb), or resin thereof.

The invention will now be described by reference to the following non-limiting Examples and the Figures.

### EXAMPLES

### Example 1—Preparation of Compound a According to Formula (I)

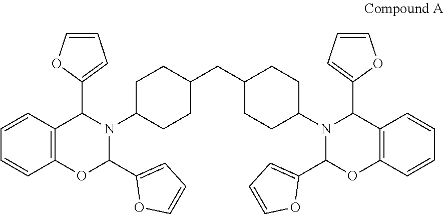 [SMILES: c1coc(C2Oc3ccccc3C(c3ccco3)N2C2CCC(CC3CCC(N4C(c5ccco5)Oc5ccccc5C4c4ccco4)CC3)CC2)c1]

184 grams of toluene was added to a 1 litre, round-bottomed flask equipped with a condenser, mechanical stirrer, addition funnel and thermometer. 384 grams [4 Mols] of unsubstituted furfuraldehyde was added and the agitator started. 210 grams [1 Mol] of 4,4-Diaminodicyclohexylmethane, was added to the flask over a 60-minute period controlling the temperature below 60° C. throughout the addition.

188 grams of phenol [2 Mols] was added in 8 aliquots maintaining the temperature below 50° C. throughout the 30-minute period. The mixture was held for a further 30 minutes before being heated to azeotrope up to 120° C. removing approximately 72 grams of water formed during the reaction.

The flask was then reconfigured for vacuum distillation and distilled under vacuum to 120° C. recovering the toluene [182 grams recovered] to yield Compound A in a liquid form.

### Example 2—Preparation of Compound B According to Formula (II)

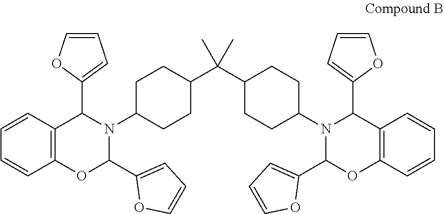 [SMILES: CC(C)(C1CCC(N2C(c3ccco3)Oc3ccccc3C2c2ccco2)CC1)C1CCC(N2C(c3ccco3)Oc3ccccc3C2c2ccco2)CC1]

184 grams toluene was added to a 1 litre, round-bottomed flask equipped with a dean and stark separator, condenser, mechanical stirrer, addition funnel and thermometer. 228 grams of bisphenol A [1 Mole] was added to the flask along with 384 grams [4 Mols] of unsubstituted furfuraldehyde and the agitator started. A nitrogen atmosphere was applied and aniline 186 grams [2 Moles] added in 4 aliquots over a period of 60 minutes. The temperature was maintained between 43-47° C. throughout the addition.

Following the addition of the aniline the flask was heated to 85° C. and held 1 hour. It was then azeotroped up to 110° C. removing approximately 71 grams water formed during the reaction. The Flask was then reconfigured for vacuum distillation and distilled under vacuum to 120° C. recovering the toluene [181 grams recovered] to yield Compound B in a liquid form.

### Example 3—Preparation of Compound C According to Formula (IIIIa)

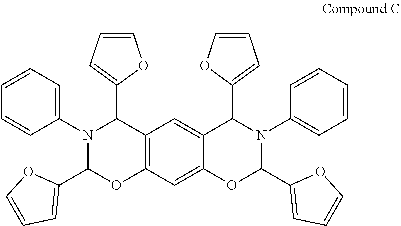 [SMILES: c1ccc(N2C(c3ccco3)Oc3cc4c(cc3C2c2ccco2)C(c2ccco2)N(c2ccccc2)C(c2ccco2)O4)cc1]

184 grams of toluene was added to a 1 litre, round-bottomed flask equipped with a dean and stark separator, condenser, mechanical stirrer, addition funnel and thermometer. 110 grams of resorcinol [1 Mole] was added to the flask along with 384 grams [4 Mols] of unsubstituted furfuraldehyde and the agitator started. A nitrogen atmosphere was applied and aniline 186 grams [2 Moles] added in 4 aliquots over a period of 60 minutes. The temperature was maintained between 45-50° C. throughout the addition.

Following the addition of the aniline the flask was heated to 85° C. and held 1 hour. It was then azeotroped up to 110° C. removing approximately 72 grams water formed during the reaction. The flask was then reconfigured for vacuum distillation and distilled under vacuum to 120° C. recovering the toluene [182 grams recovered] to yield Compound C in a liquid form.

### Example 4—Preparation of Compound D According to Formula (I)

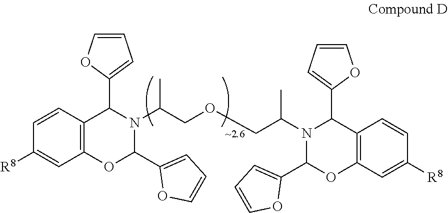

Wherein R⁸ is independently selected from a —C₁₅H₃₁ straight chain alkyl group, —C₁₅H₂₉ straight chain alkenyl group, —C₁₅H₂₇ straight chain alkenyl group, and —C₁₅H₂₅ straight chain alkenyl group.

184 grams of toluene was added to a 1 litre, round-bottomed flask equipped with a dean and stark separator, condenser, mechanical stirrer, addition funnel and thermometer. 230 grams of Jeffamine D230 (RTM) [1 Mole] was added to the flask along with 384 grams [4 Mols] of unsubstituted furfuraldehyde and the agitator started. A nitrogen atmosphere was applied and 660 grams [2 Moles] of cardanol added. The temperature was maintained <75° C. throughout the additions.

Following the addition of the cardanol the flask was heated to 85° C. and held 1 hour. It was then azeotroped up to 110° C. removing approximately 71 grams of water formed during the reaction. The flask was then reconfigured for vacuum distillation and distilled under vacuum to 120° C. recovering the toluene [180 grams recovered] to yield Compound D in a liquid form.

### Example 5—Comparison of Physical Properties Against Known Benzoxazines

TABLE 4
Comparison of the properties of Compounds A to D with those of a
convention benzoxazine (Cardarez R111)

| | | Plate | Viscosity | Tg/° C. | |
|---|---|---|---|---|---|
| | | gel at | at | (cured at | |
| | | 200° C./ | 25° C./ | 180° C. for | |
| | Colour | minutes | Pa · S | 2 hours, | Bio- |
| | (ASTM | (ASTM | (ASTM | and then | carbon |
| | D1544- | D3056- | D2556- | 200° C. for | content/ |
| Compound | 04(2018)) | 14(2018)) | 14(2018)) | 4 hours) | % |
| Compound A | 18 (black) | 6:55 | 115 | 190 | 44 |
| Compound B | 18 (black) | 7:22 | 215.7 | 128 | 43 |
| Compound C | 18 (black) | 7:18 | 345.2 | 114 | 53 |
| Compound D | 18 (black) | 9:18 | 11.8 | 123 | ~85 |
| Cardarez R111 | 18 (black) | 12:00 | 3* | 160 | 0 |
| (RTM) | | | | | |

*Solution viscosity is undertaken as a 50% weight/weight solution in toluene as Cardarez R111 (RTM) is a solid.

The results in Table 4 demonstrate the advantages of the benzoxazine compounds of the invention. The benzoxazines of the present invention provide a significant bio-carbon contents, exceeding 40% in each case, meaning that the compounds can help meet environmental targets in the applications in which they are deployed and are produced using renewal feedstocks.

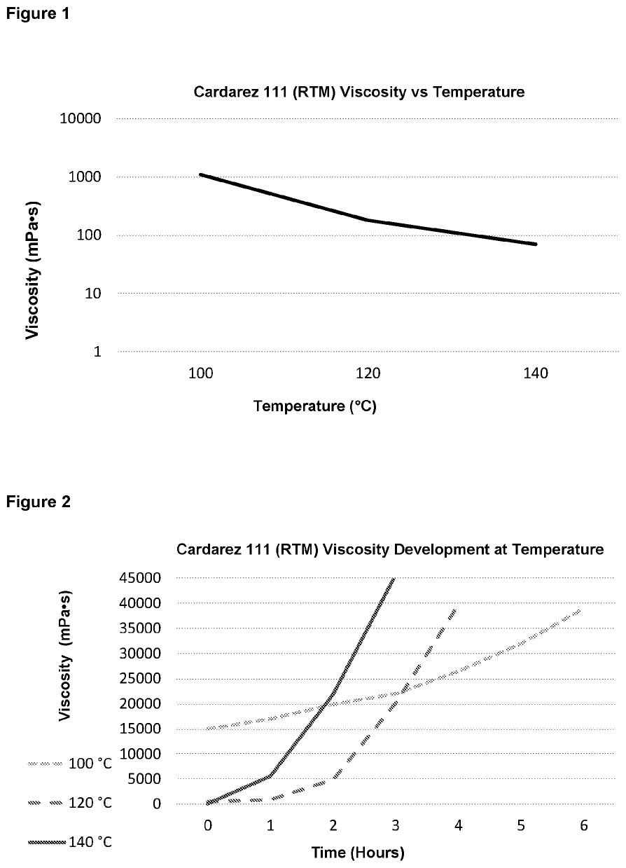

The benzoxazines of the present invention also have advantageously low viscosities which provides for easier handling during preparation, storage and use, as well as reducing the need for diluents or solvents in a resin composition. Conventional benzoxazine, Cardarez R111 (RTM), is a solid, and as such no directly comparable viscosity measurement can be taken at 25° C. However, FIG. 1 demonstrates the viscosity (mPa·s) v temperature (° C.) of Cardarez R111 (RTM), using ASTM D4287-00(2019). Due to the solid nature of Cardarez R111 (RTM) viscosity measurements must be taken at high temperatures in order to melt the Cardarez R111 (RTM). It can be seen from FIG. 1 that temperatures as high as 120° C. to 140° C. are required before the viscosity of Cardarez R111 (RTM) is at a comparable order of magnitude to the viscosities of compounds A to D at 25° C.

FIG. 2 demonstrates viscosity (mPa·s) development over time (hours) of Cardarez R111 (RTM) at 100° C., 120° C. and 140° C. Cardarez R111 (RTM) is a thermoset and reactivity rate [devoid of catalysis or co-polymer] is temperature dependent and depicted in FIG. 2. Cardarez R111 (RTM) stability evaluation undertaken with a 50 gram mass in all cases. Viscosity figures quoted are evaluated and detailed at the temperature that the material was held. Viscosity evaluation was undertaken using a cone and plate viscometer [CPV] and the test temperature for viscosity evaluation is the same as the designated stability test temperature[s].

As will be appreciated, each of the compounds of the invention exhibits a significantly shorter gel time (as measured by ASTM D3056-14(2018)) in comparison to conventional benzoxazine, Cardarez R111. Relatively slow curing times are a known deficiency associated with conventional benzoxazines. The benzoxazines of the present invention provide surprising improvements in gel time, indicating that the benzoxazines of the present invention exhibit greater reactivity, leading to improvements in process efficiency in applications where benzoxazines are employed.

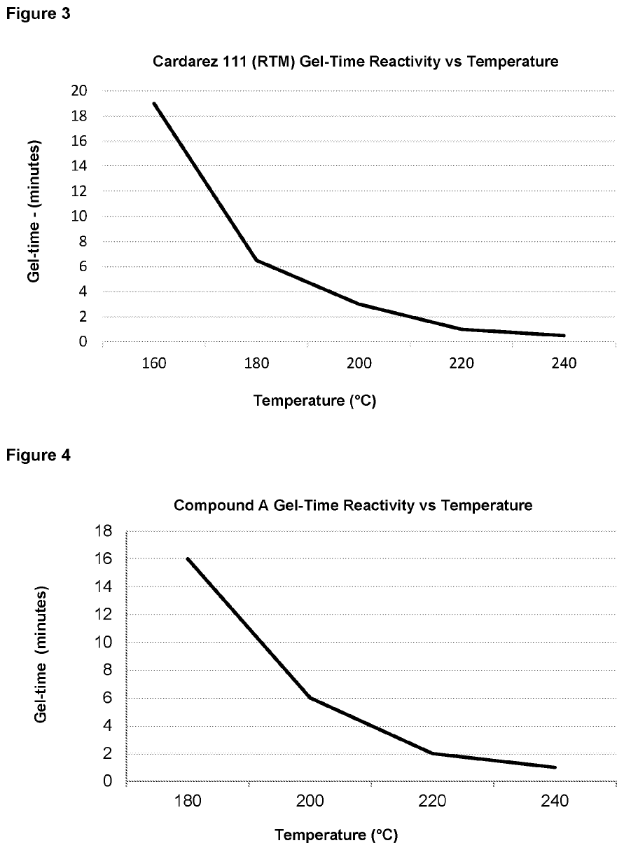

FIG. 3 and FIG. 4 compare the gel-time reactivity (gel time/minutes) undertaken with a 1 gram mass of Cardarez R111 (RTM) and Compound A, respectively. Cure determination by plate gel at specified temperatures (° C.). The gel time is representative of the progress of curing by self-polymerisation. No catalyst or co-reactant was used. FIGS. 3 and 4 are in contrast to the plate gel data (ASTM D3056-14(2018)) shown in Table 4. The ASTM D3056-14(2018) method used in Table 4 uses a catalyst, whereas the experiments shown in FIGS. 3 and 4 were performed in the absence of a catalyst. As can be seen, Cardarez R111 (RTM) is less amenable to catalysis as compared to the compounds of the invention which are particularly well suited to catalysed curing. The addition of the catalyst to the compounds of the present invention provides for a much greater reduction in gel time. Without being bound to any particular theory, it is expected that the furan groups on the benzoxazine ring assist in co-ordinating the catalyst to bring it into closer proximity with the benzoxazine ring to stimulate ring opening.

The results in Table 4 also show that glass transition temperatures compare favourably with the conventional benzoxazine (Cardarez R111) or, in the case of Compound A, are improved over the conventional benzoxazine.

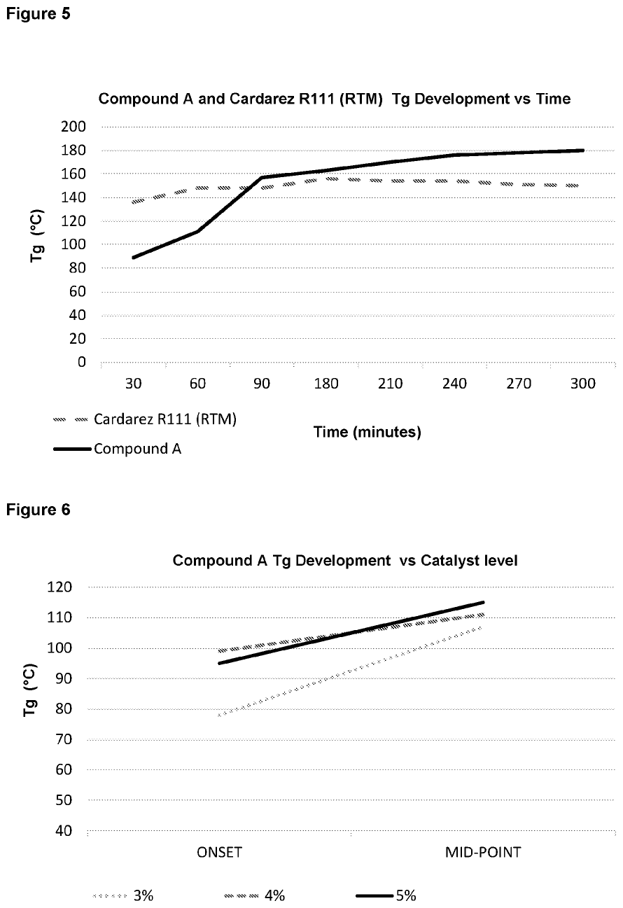

FIG. 5 demonstrates the Tg development (° C.) of thermosets cured at 180° C. vs time (minutes) for a Compound A thermoset and a comparative Cardarez R111 (RTM) thermoset. As can be seen, Compound A provides superior Tg development over time and thus is able to produce thermosets of a higher Tg once sufficient cure time is applied. As would be appreciated, cure times of 90 minutes or greater are commonly used in the art for various applications.

It has also been demonstrated that the benzoxazine compounds of the present invention are particularly well suited to curing with a ferric chloride catalyst, although any suitable Lewis acid catalyst may also be used. FIG. 6 demonstrates the Tg development (° C.) of Compound A during curing at 140° C. over 1 hour with 3%, 4% or 5% by weight of a ferric chloride catalyst. Increased concentration of catalyst can clearly be seen to accelerate curing in order to rapidly provide thermosets of higher Tg.

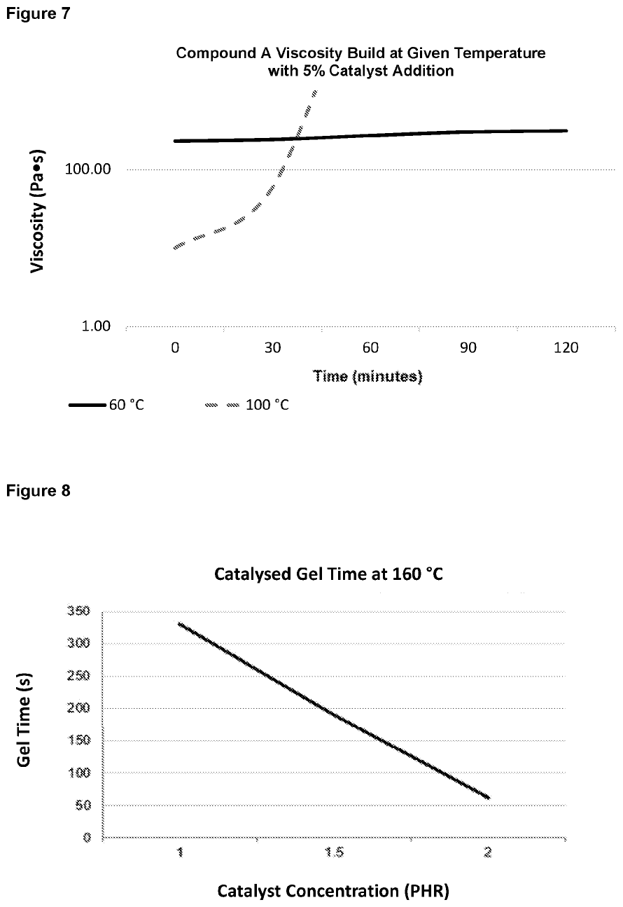

Similarly, FIG. 7 demonstrates the Compound A viscosity build (Pa s) vs time (minutes) at 60° C. and 100° C., with 5% ferric chloride catalyst. The 5% ferric chloride allows for curing to take place at significantly lower temperatures. Cardarez R111 (RTM) is a solid and so must be heated to a high temperature and melted before a catalyst may be mixed with the Cardarez R111 (RTM). This means that the catalysis of a solid benzoxazine such as Cardarez R111 (RTM) requires addition of the catalyst at high temperature followed by mixing before it can be applied in the desired use. This risks premature curing and causes difficulty in handling and storage. The benzoxazine compounds of the present invention, however, are liquids at room temperature (e.g. 25° C.) and so may be mixed with a catalyst at comparatively low temperature without the risk of premature curing.

As can be seen from FIG. 7, the viscosity build is very slow at 60° C., which demonstrates the utility for storage and handling of Compound A pre-mixed with catalyst at low temperature. Additionally, rapid curing can nevertheless be achieved, once desired, by heating to a higher temperature, e.g. 100° C. This is not possible for a solid benzoxazine which cannot be pre-mixed with a catalyst prior to melting at high temperature.

### Example 6—Preparation of Reinforced Material

A prepreg composite material was provided, in which a reinforcement fibre is pre-impregnated with a resin composition comprising Compound A. Compound A is a viscous liquid and can be commercially processed via hot-melt or solvated and processed through conventional methods.

The material was used to make a composite part produced by a standard prepreg stage and pressing at a consolidation pressure 80 bar/cm². Each composite part was generated using glass fabric with fibre density-binder ratio of 60:40 by weight: E-glass fabric, Style 7628 Finish phenolic compatible.

The cure schedule employed was heating at a rate of 8° C./minute from RT up to 180° C., followed by holding at 180° C. for 3 hours under a consolidation pressure of 80 bar/cm².

The resultant composite panel had excellent surface appearance, free from defects such as porosity. The resultant product is suitable for use with a variety of reinforcements finding application in numerous areas including but not limited to, aircraft interiors and flooring, aerospace components, cargo liners, automotive parts, ballistic components, electrical laminates, fire resistant laminates and panels, sporting goods, train components, and tooling.

### Example 7—Sample Laminate Properties

The physical properties, and chemical resistance properties of a reinforced material according to Example 6 were measured and compared to a comparative reinforced material produced by the same method of Example 6 using a known benzoxazine (Cardarez R111 (RTM))

TABLE 5
Reinforced material physical properties comparison

| Physical Property | Cardarez R111 (RTM) | Compound A |
|---|---|---|
| Tg/° C. | 160 | 190 |
| Flexural strength/MPa | 580 | 550 |
| Flexural modulus/GPa | 27 | 25 |
| Impact strength/KJM² | 48 | 50 |
| UL 94 evaluation | — | V0 |

Chemical resistance of the reinforced material of the invention vs a comparative reinforced material was determined by weight change following submersion of the materials. The samples were submerged in the given chemical for 4 weeks at 25° C.

TABLE 6
Reinforced material chemical resistance comparison

| Chemical | Cardarez R111 (RTM) | Compound A |
|---|---|---|
| submersion | Weight gain/loss/% | Weight gain/loss/% |
| Acetone | −7.00 | +4.58 |
| Butyl acetate | −1.23 | +3.36 |
| Ethylene diamine | Destroyed | Destroyed |
| Methanol | −0.64 | +5.61 |
| Nitric acid 20% | −0.74 | +0.47 |
| Sulphuric acid 50% | −3.08 | +1.24 |
| Xylene | −0.42 | +1.39 |

As can be seen from Table 5, the reinforced material made with compound A, has comparable strength and flexibility to that of a reinforced material produced with Cardarez R111 (RTM). The reinforced material made with Compound A is also shown to have a high degree of fire resistance. As can be seen from Table 6, the reinforced material made with Compound A, also has comparable chemical resistance to that of a reinforced material produced with Cardarez R111 (RTM). A very similar degree of weight change (indicative of chemical degradation) is generally observed as a result of chemical exposure.

The present invention thus allows for the provision of reinforced materials comprising a high degree of bio-carbon from renewable stocks without negatively impacting the physical properties or chemical resistance.

### Example 8—Effects of Catalysis

### Effect of Catalyst on Gel Time

The effects of catalysis on gel time were investigated by measuring the gel time of a solution of 90% Compound A and 10% acetone with various concentrations of a curing catalyst at 160° C. The catalyst used was BCl₃ octyl dimethylamine complex and its relative concentration was measured in parts per hundred (PHR) with respect to Compound A.

A solution of BCl₃ octyl dimethylamine complex in ethanol was added to the solution of Compound A and mixed for 5 minutes with a slow rate agitator to form a homogenous solution without introducing air. The combined material had a viscosity of 1180 mPa·s at 25° C. The gel times of Compound A in combination with concentrations of 1, 1.5 and 2 PHR of BCl₃ octyl dimethylamine complex at 160° C. were found to be approximately 320 seconds, 180 seconds, and 60 seconds, respectively. As would be expected, increasing the concentration of catalyst decreases the gel time, and these results are shown graphically in FIG. 8.

### Preparation of Reinforced Material Using a Catalyst

A combined solution of 90% Compound A and 10% acetone combined with 1 PHR of BCl₃ octyl dimethylamine complex was used to prepare a prepreg material. The combined solution was applied via a conventional process of impregnation of an E-glass fabric cloth (Style 7628-finish phenolic compatible) having a fibre density of 70% by weight, with the solution followed by driving off the solvent and partially advancing the resin to form a partially cured state or state of advancement/cure that allows the sheets to have sufficient strength so that they can be laid up as a laminate (prepreg layer stack), but not so advanced that they are rigid or fail to flow when pressed under heat and pressure. The sheets were cut and then aligned so they can be consolidated, and the cure schedule employed was heating at a rate of 8° C./minute up to 170° C., followed by heating at 170° C. for two hours, followed by heating at a rate of 8° C./minute up to 190° C. under a consolidation pressure of 80 bar/cm². The resulting laminate had the following properties:

TABLE 7
Catalysed reinforced material physical properties

| | Compound A | Testing |
|---|---|---|
| Physical Property | (1 PHR Catalyst) | Method |
| Density/g/cm³ | 1.95 | ISO 1183 |
| Flexural strength/MPa | 500 | ISO 178 |
| E-modulus (flexural strength)/ | 35 | ISO 178 |
| GPa | | |
| Tensile strength/MPa | 400 | ISO 527 |
| Compression strength | 500 | ISO 604 |
| perpendicular to lamination/ | | |
| MPa | | |
| Breakdown voltage/KV | 75 | IEC 60243-1 |
| (d = 25 mm)* | | |
| Flammability 5 mm | V0** | UL 94 |

*(d = 25 mm) denotes that both electrodes have a 25 mm diameter.
**V0 = Burning stops within 10 seconds after two applications of ten seconds each of a flame/vertical burn to a test bar. No flaming drips are observed.

Thus, the present invention is highly compatible with curing catalysts and may readily provide reinforced materials having desirable properties derived from the compounds described herein, prepared with the assistance of a curing catalyst.

1. A compound of formula (I): 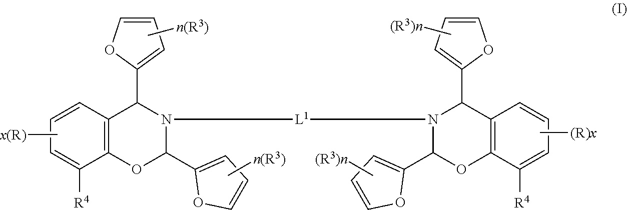
wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein L¹ is a divalent hydrocarbyl linker comprising 1 to 50 carbon atoms;
wherein x is independently 0, 1, 2 or 3; and
wherein n is independently 0, 1, 2 or 3.

2. The compound of claim 1, wherein one or both benzene rings of the benzoxazines are substituted with R groups at the 3-position and/or the 5-position, relative to the oxygen of the benzoxazine ring.

3. The compound of claim 1, wherein both x values ≤2, and/or wherein both benzene rings of the benzoxazines are substituted with the same R group(s).

4. The compound of claim 1, wherein L¹ comprises 5 to 20 carbon atoms.

5. The compound of claim 1, wherein L¹ comprises at least one of:
i) a heteroatom selected from nitrogen, oxygen, sulphur and silicon;
ii) a double or triple carbon-carbon bond; or
iii) an aromatic ring, heteroaromatic ring, and/or non-aromatic ring.

6. The compound of claim 1, wherein L¹ is derived from the reaction of a diamine NH₂-L¹-NH₂, when NH₂-L¹-NH₂ is selected from: ethylene diamine, diethylene triamine, triethylene tetramine, hexamethylene diamine, 2,2,4-trimethyl hexamethylene diamine, 2,4,4-trimethyl hexamethylene diamine, a Jeffamine, metaxylylene diamine, 1,3-bis(aminomethyl) cyclohexane, benzene-1,4-diamine, isophorone diamine, diaminocyclohexane, 4-Cyclohexene-1,2-diamine, 3-Hexyne-1,6-diamine, bis-(p-aminocyclohexyl) methane or methylene dianiline.

7. The compound of claim 1, wherein the compound has the structure: 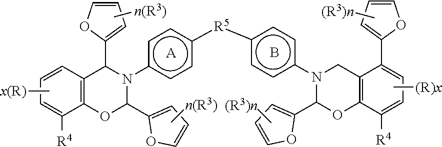
wherein ring A and ring B are independently either aromatic or saturated 6 membered carbon rings;
wherein R⁵ is a divalent C₁ to C₁₀ alkyl group and
wherein R, R³, x, and n are independently as defined in claim 1.

8. A compound of formula (II): 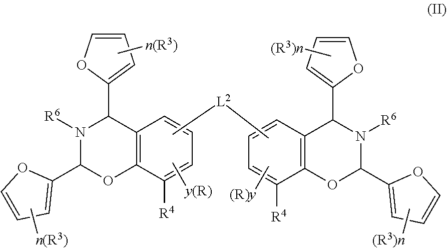
wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein L² is a direct bond, a divalent group selected from: C(O), O, S, S(O), S(O)₂, or a hydrocarbyl linker comprising 1 to 50 carbon atoms;
wherein y is independently 0, 1, or 2; and
wherein n is independently 0, 1, 2 or 3.

9. The compound of claim 8, wherein L² is attached at the 4-position, relative to the benzoxazine oxygen; and/or wherein L² is selected from S(O)₂, CH₂, C(CH₃)₂, or a divalent benzene ring.

10. The compound of claim 8, wherein both y values ≤1, and/or wherein both benzene rings of the benzoxazines are substituted with the same R group(s), more preferably wherein both y values=0.

11. A compound of either formula (IIIa) or formula (IIIb): 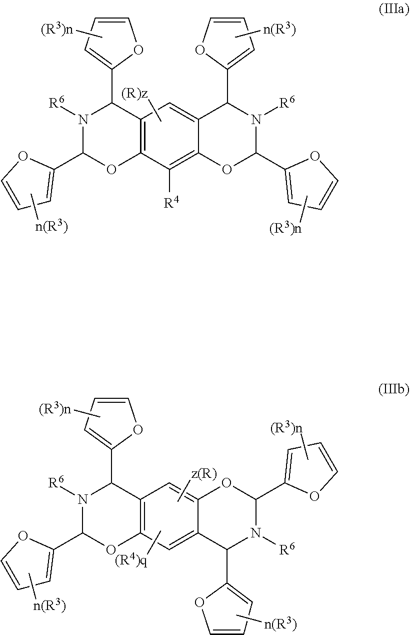
wherein R is selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently a monovalent group selected from —H, —OH, or a hydrocarbyl group comprising 1 to 50 carbon atoms;
wherein z is 0 or 1;
wherein q=2−z; and
wherein n is independently 0, 1, 2 or 3.

12. The compound of claim 1, wherein each R group is independently selected from: C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —O(CO)H, —O(CO)R¹, —NH(CO)H, —NH(CO)R¹, —NR¹(CO)H, —NR¹(CO)R¹, —SH, —SR¹, —SiR¹₃, —F, —Cl, —Br, —I, C₆ to C₁₄ aryl optionally substituted with one or more R² group, and C₂ to C₉ heteroaryl optionally substituted with one or more R² group;
wherein R¹ is independently selected from: C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀, heterocycloalkyl, C₁ to C₂₀ alkoxy, C₁ to C₂₀ alkylamino, C₆ to C₁₄ aryl, and C₂ to C₉ heteroaryl; and
wherein R² is independently selected from: C₁ to C₂₀ alkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —F, —Cl, —Br, or —I.

13. The compound of claim 1, wherein each R group is independently selected from: C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, —OH, —OR¹, —NH₂, —NHR¹, —NR¹₂, C₆ to C₁₄ aryl optionally substituted with one or more R² group, or C₂ to C₉ heteroaryl optionally substituted with one or more R² group;
wherein R¹ is independently selected from: C₁ to C₂₀ alkyl, C₃ to C₂₀ cycloalkyl or C₂ to C₂₀ alkenyl;
wherein R² is independently selected from: C₁ to C₂₀ alkyl, —OH, —NH₂, —OC₁ to C₂₀ alkyl, —NHC₁ to C₂₀ alkyl or —N(C₁ to C₂₀ alkyl)₂, preferably —OH.

14. The compound of claim 1, wherein R³ is independently selected from: C₁ to C₆ alkyl, C₁ to C₆ alkoxy, C₁ to C₆ alkyloxy, and —OH.

15. The compound of claim 1, wherein all n values ≤1, and/or wherein all R³ groups are the same, and/or wherein all R³ groups are attached at the position ortho to the furan oxygen.

16. The compound of claim 8, wherein R⁶ is independently selected from: —H, —OH, C₁ to C₂₀ alkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, C₆ to C₁₄ aryl, and C₂ to C₉ heteroaryl; wherein R⁶ is optionally substituted with one or more R⁷ groups;
Wherein R⁷ is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl, and C₂ to C₉ heteroaryl.

17. A process for preparing a compound of formula (I) according to claim 1, said method comprising:
i) providing a diamine NH₂-L¹-NH₂ wherein L¹ is as defined in claim 1;
ii) adding at least one furfuraldehyde substituted with n R³ groups, and at least one phenolic compound substituted with x R groups, wherein R, R³, n and x are as defined in claim 1, with the proviso that both positions ortho to the phenolic OH group are unsubstituted on the at least one phenolic compound; and
iii) forming a compound of formula (I).

18. The process of claim 17, wherein the molar ratio of the diamine to the at least one furfuraldehyde is from 1:2 to 1:20 and/or wherein the molar ratio of the diamine to the at least one phenolic compound is from 1:1 to 1:5.

19. A process for preparing a compound of formula (II), (IIIa), or (IIIb); 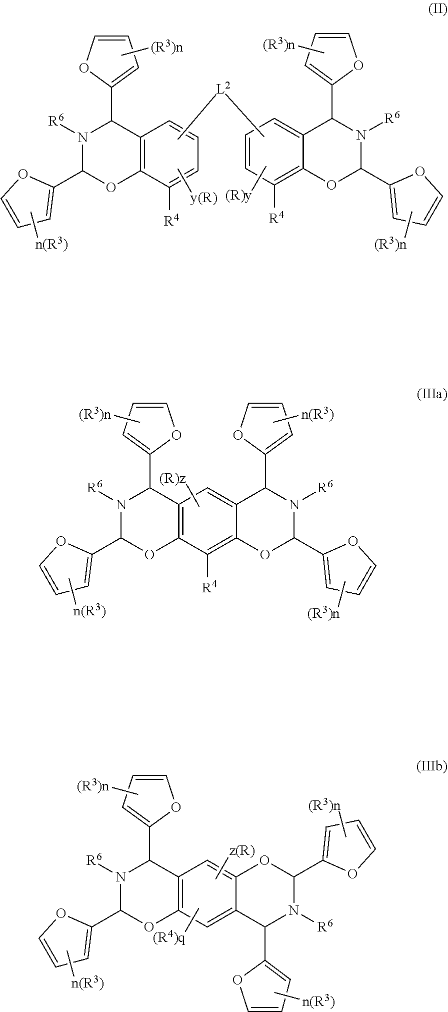
wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₁ to C₂₀ alkyl-C₃ to C₁₀ heterocycloalkyl, C₁ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₁ to C₂₀ alkyl-C₃ to C₂₀ heterocycloalkyl, C₁ to C₂₀ alkoxy, C₁ to C₂₀ alkylamino, C₆ to C₁₄ aryl, and C₂ to C₉ heteroaryl;
wherein R² is independently selected from: C₁ to C₂₀ alkyl, C₁ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)O—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently monovalent group selected from —H, —OH or a hydrocarbyl group comprising 1 to 50 carbon atoms;
wherein L² is a direct bond, a divalent group selected from: C(O), O, S, S(O), S(O)₂, or a hydrocarbyl linker comprising 1 to 50 atoms;
wherein y is independently 0, 1 or 2;
wherein z is 0 or 1;
wherein q=2−z; and
wherein n is independently 0, 1, 2 or 3;
said method comprising:
i) providing: A) a bisphenol compound comprising a linker L² and substituted with y R groups on each benzene ring of the bisphenol compound; B) a benzene-1,4-diol compound substituted with z R groups; or C) a benzene-1,3-diol optionally substituted with 1 R group at the 5-position;
ii) contacting the compound from step i) with at least one furfuraldehyde substituted with n R³ groups, and at least one primary amine substituted with one R⁶ group; and
iii) forming a compound of formula (II), (IIIa), or (IIIb).

20. The process of claim 19, wherein the molar ratio of the bisphenol compound or benzenediol to the at least one furfuraldehyde is from 1:2 to 1:20 and/or wherein the molar ratio of the bisphenol compound or benzenediol to the at least one primary amine is from 1:1 to 1:5.

21. The process of claim 17, wherein either; the diamine or at least one primary amine, is contacted with the at least one furfuraldehyde prior to the addition of either; the at least one phenolic compound, or the bisphenol compound or benzenediol, respectively.

22. The process of claim 17, wherein either; the diamine or at least one primary amine, is contacted with a portion of the at least one furfuraldehyde prior to the addition of either; the at least one phenolic compound, or the bisphenol compound or benzenediol, respectively, wherein the remaining furfuraldehyde is added after and/or simultaneously to addition of either; the at least one phenolic compound, or the bisphenol compound or benzenediol, respectively.

23. The process of claim 17, wherein either; the diamine or at least one primary amine, is contacted with either; the at least one phenolic compound, or the bisphenol compound or benzenediol, respectively, and the at least one furfuraldehyde simultaneously.

24. The process of claim 17, wherein two different phenolic compounds or two different primary amines are added separately; or wherein only one phenolic compound or only one primary amine is added.

25. A resin composition comprising the compound of claim 1.

26. (canceled)

27. (canceled)

28. A method for preparing a reinforced material, said method comprising:
a) contacting a material to be reinforced with a compound of formula (I), (II), (IIIa), and (IIIb); 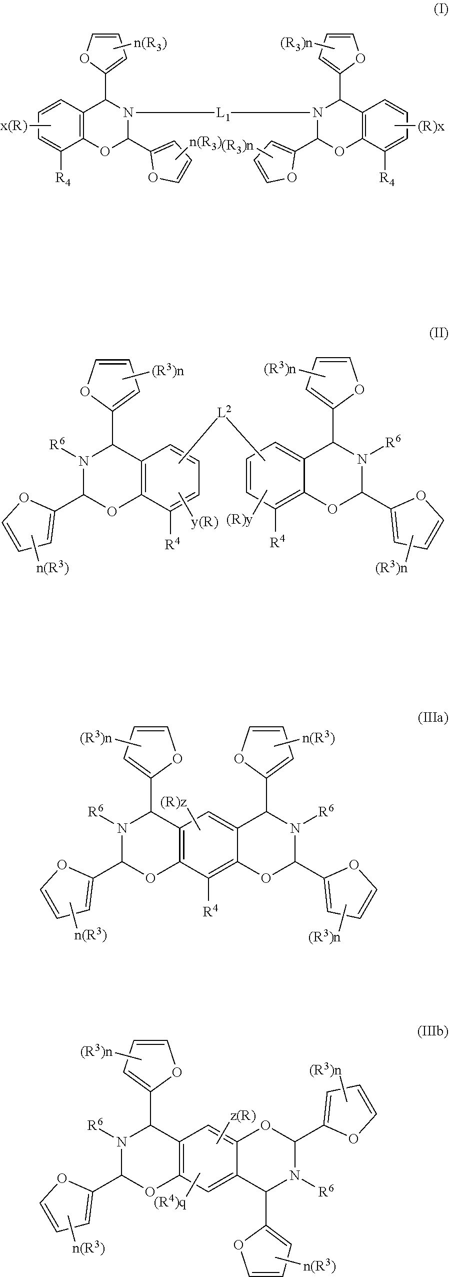
wherein each R group is independently selected from: C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₂₀ cycloalkyl, C₃ to C₂₀ alkyl-C₃ to C₁₀ heterocycloalkyl, C₃ to C₂₀ alkyloxy, C₁ to C₂₀ alkylamino, —OH, —OR¹, —NH₂, —NHR¹, —NR¹₂, —C(O)OH, —C(O)OR¹, —C(O)NH₂, —C(O)NHR¹, —C(O)NR¹₂, —O(CO)H, —O(CO)R¹, —NH(CO)H, —NH(CO)R¹, —NR¹(CO)H, —NR¹(CO)R¹, —SH, —SR¹, —SO₂H, —SO₂R¹, —SO₃R¹, —SO₃H, —SiR¹₃, —NO₂, —CN, —F, —Cl, —Br, —I, C₆ to C₁₄ aryl optionally substituted with one or more R² group, and C₂ to C₉ heteroaryl optionally substituted with one or more R² group;
wherein each R¹ is independently selected from C₁ to C₂₀ alkyl, C₁ to C₂₀ haloalkyl, C₃ to C₂₀ cycloalkyl, C₃ to C₂₀ heterocycloalkyl, C₂ to C₂₀ alkenyl, C₂ to C₂₀ alkynyl, C₁ to C₂₀ alkyl-C₆ to C₁₄ aryl, C₁ to C₂₀ alkyl-C₂ to C₉ heteroaryl, C₁ to C₂₀ alkyl-C₃ to C₁₀ cycloalkyl, C₃ to C₂₀ alkyl-C₃ to C₂₀ heterocycloalkyl, C₁ to C₂₀ alkoxy, C₃ to C₂₀ alkylamino, C₆ to C₁₄ aryl, and C₂ to C₉ heteroaryl;
wherein R² is independently selected from: C₁ to C₂₀ alkyl, C₃ to C₂₀ haloalkyl, C₃ to C₂₀ cycloalkyl, C₂ to C₂₀ alkenyl, C₂ to C₂₀ alkynyl, —OH, —NH₂, —OC₁ to C₂₀ alkyl, —NHC₁ to C₂₀ alkyl, —N(C₁ to C₂₀ alkyl)₂, —NO₂, —CN, —F, —Cl, —Br, and —I;
wherein R³ is independently selected from: C₁ to C₆ alkyl, C₁ to C₆ alkenyl, C₁ to C₆ alkoxy, C₁ to C₆ alkyloxy, —C(O)OH, —C(O)OC—C₁ to C₆ alkyl, C₁ to C₆ alkyl-(O)O—H, C₁ to C₆ alkyl-(O)O—C₁ to C₆ alkyl, and —OH;
wherein R⁴ is hydrogen;
wherein R⁶ is independently monovalent group selected from —H, —OH or a hydrocarbyl group comprising 1 to 50 carbon atoms;
wherein L¹ is a divalent hydrocarbyl linker comprising 1 to 50 carbon atoms;
wherein L² is a direct bond, a divalent group selected from: C(O), O, S, S(O), S(O)₂, or a hydrocarbyl linker comprising 1 to 50 atoms;
wherein x is independently 0, 1, 2 or 3;
wherein y is independently 0, 1 or 2;
wherein z is 0 or 1;
wherein q=2−z; and
wherein n is independently 0, 1, 2 or 3;
b) allowing said compound to self-polymerise to form a reinforced material.

29. A reinforced material prepared, or preparable, by the method of claim 28.
